(19) United States
(12) Patent Application Publication
(10) Pub. No.: US 2024/0226342 A1
(43) Pub. Date: Jul. 11, 2024

(54) REACTIVE OXYGEN SPECIES IMAGING AGENTS

(71) Applicants: Vijay Sharma, St. Louis, MO (US); Lakshminarayana Satham, St. Louis, MO (US); Jothilingam Sivapackiam, St. Louis, MO (US)

(72) Inventors: Vijay Sharma, St. Louis, MO (US); Lakshminarayana Satham, St. Louis, MO (US); Jothilingam Sivapackiam, St. Louis, MO (US)

(73) Assignee: Washington University, St. Louis, MO (US)

(21) Appl. No.: 18/529,171

(22) Filed: Dec. 5, 2023

Related U.S. Application Data
(60) Provisional application No. 63/386,033, filed on Dec. 5, 2022.

(51) Int. Cl. A61K 51/04

(52) U.S. Cl. CPC A61K 51/0459

(57) ABSTRACT

The present disclosure provides compositions and methods for reactive oxygen species imaging agents. Compositions include a reactive oxygen species (ROS) imaging agent according to any one of Formulas (I-VI)ₐ and (I-VII)_{b}. Methods of detecting ROS in a subject include administering to the subject an effective amount of a composition comprising a reactive oxygen species (ROS) imaging agent according to any one of Formulas (I-VI)ₐ and (I-VII)_{b}, and exposing the subject to an imaging modality such as PET or CT. Administering the composition comprising the ROS imaging agent results in penetration of the ROS imaging agent into a membrane, oxidation of the ROS imaging 10 agent by ROS, and trapping of the ROS imaging agent in a cell membrane and intracellular compartments.

### CROSS-REFERENCE TO RELATED APPLICATIONS

This application claims priority from U.S. Provisional Application Ser. No. 63/386,033 filed on 5 Dec. 2022, which is incorporated herein by reference in its entirety.

### STATEMENT REGARDING FEDERALLY SPONSORED RESEARCH OR DEVELOPMENT

This invention was made with government support under AG064937, AG050263, and EB025815 awarded by the National Institutes of Health. The government has certain rights in the invention.

### MATERIAL INCORPORATED-BY-REFERENCE

Not applicable.

### FIELD

The present disclosure generally relates to detection of reactive oxygen species (ROS) using imaging agents and methods of use thereof.

### SUMMARY

Among the various aspects of the present disclosure is the provision of reactive oxygen species imaging agents.

In one aspect of the present disclosure, a composition is provided. The composition comprises a reactive oxygen species (ROS) imaging agent according to any one of Formulas (I-VI)ₐ and (I-VII)_{b}:

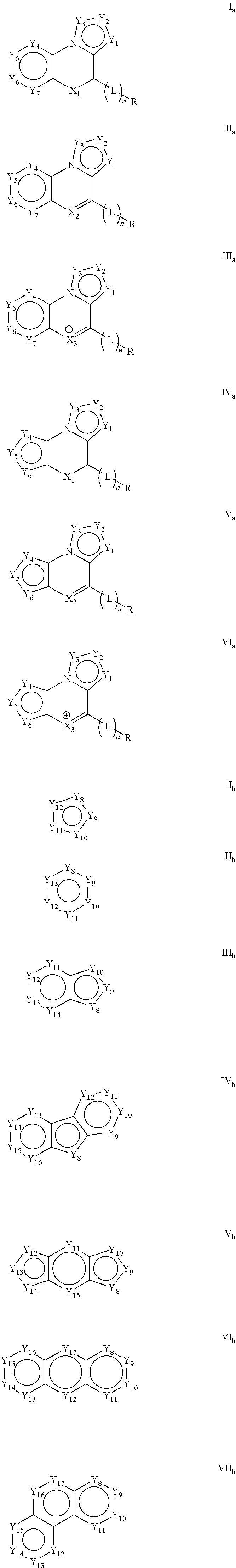

wherein:
X₁ and X₃ are independently NR¹, O, S, or PR²;
X₂ is N or P;
L is

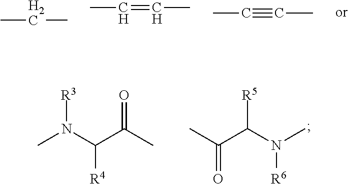

n is 0, 1, 2, 3, or 4;
R is H, CH₃, alkyl chain (linear or branched, number of carbons can be 1-4), cycloalkyl (ring size can be 3-7), —OR⁷, NH₂, NHR⁸, NR⁹R¹⁰, SH, SR¹¹, PH₂, PHR¹², PR¹³R¹⁴, halo, CN, NO₂, COOH, COOR¹⁵, CHO, COR¹⁶, CONH₂, CONHR¹⁷, CONR¹⁸R¹⁹, SOR²⁰, SO₂R²¹, SO₃R²², or a chelator core;
Y₁-Y₁₇ are independently CH, CR²³ (wherein R²³ can be alkyl, alkenyl, alkynyl), —OH, —OR²⁴, NH₂, NHR²⁵, NR²⁶R²⁷, SH, SR²⁸, PH₂, PHR²⁹, PR³⁰R³¹, halo, BR³², BR³³R³⁴, CN, NO₂, COOH, COOR³⁴, CHO, COR³⁶, CONH₂, CONHR³⁷, CONR³⁸R³⁹, SOR⁴⁰, SO₂R⁴¹, SO₃R⁴², a chelator core, CO, COO, COS, CONR⁴³, N, NH, NR⁴⁴, NO, O, S, SO, SO₂, BR⁴⁵, or BR⁴⁶R⁴⁷; and
R¹-R⁴⁷ are independently H, halo, C₁-C₁₂ linear alkyl, C₂-C₁₂ linear alkenyl, C₂-C₁₂ linear alkynyl, C₃-C₁₂ branched chain alkyl, C₃-C₁₂ branched chain alkenyl, C₃-C₁₂ branched chain alkynyl and C₃-C₇ cycloalkyl, or (hetero)aryl.

In some embodiments, R comprises a chelator core selected from NOTA, NODA, DOTA, DTPA, or Triglycine; H is T or ³H; and/or halo is Cl, F, Br, I, ¹⁸F, ⁷⁵Br, ⁷⁶Br, ⁷⁷Br, ¹²³I, ¹²⁴I, ¹²⁵I or ¹³¹I.

In some embodiments, the ROS imaging agent comprises a chelator core and a metal radionuclide. In some embodiments, the metal radionuclide is an ion of gallium-67 (⁶⁷Ga), gallium-68 (⁶⁸Ga), an unlabeled gallium, or a paramagnetic metal which includes an ion of ⁶⁷Ga, an ion of ⁶⁸Ga, an ion of an unlabeled gallium, -indium-111 (¹¹¹In), -iron-52 (⁵²Fe), iron-59 (⁵⁹Fe), -copper-62 (⁶²Cu), -copper-64 (⁶⁴Cu), -thallium-201 (²⁰¹Tl) -technetium-99m (^{99m}Tc), -technetium-94m (^{94m}Tc), -rhenium-188 (¹⁸⁸Re), -rubidium-82 (⁸²Rb), -strontium-92 (⁹²Sr), -yttrium-86 (⁸⁶Y) or yttrium-90 (⁹⁰Y), -zirconium-86 (⁸⁶Zr) or -zirconium-89 (⁸⁹Zr), -aluminium fluoride-18 (Al¹⁸F), a paramagnetic metal ion, a transition metal, or a lanthanide metal ion.

In some embodiments, the ROS imaging agent comprises a radiolabel or radionuclide. In some embodiments, the ROS imaging agent comprises the radiolabel ¹⁸F or ¹¹C.

In some embodiments, the ROS imaging agent is selected from the group consisting of:

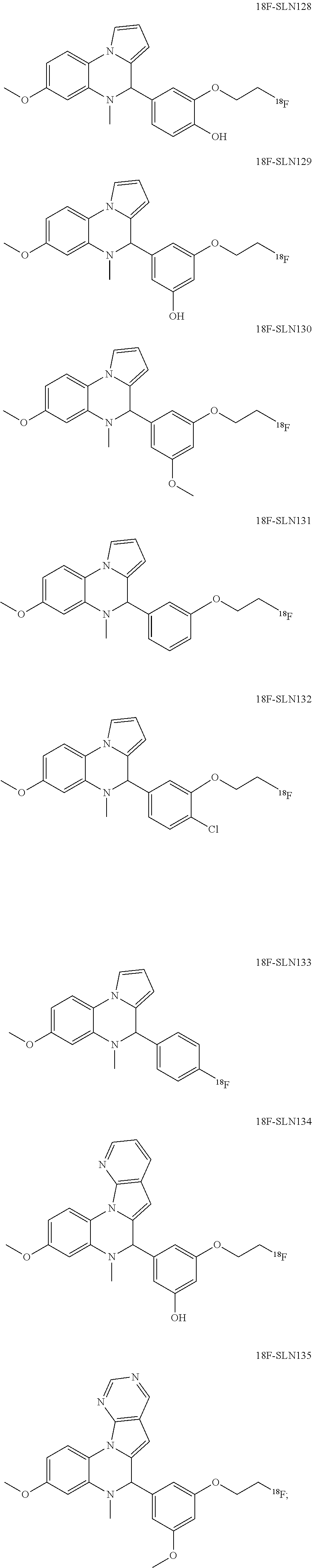

or
a pharmaceutically acceptable salt, solvate, polymorph, tautomer, prodrug, or analog thereof.

In another embodiment of the present disclosure, a method of detecting ROS in a subject is provided. The method comprises administering to the subject an effective amount of a composition comprising a reactive oxygen species (ROS) imaging agent according to any one of Formulas (I-VI)ₐ and (I-VII)_{b}:

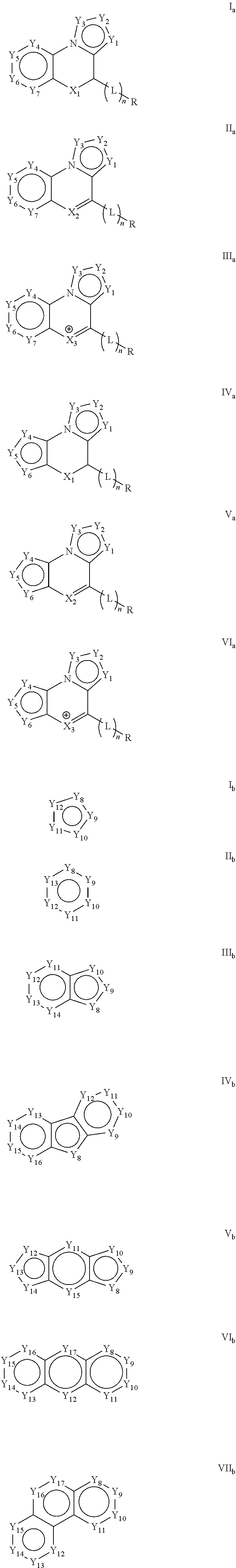

wherein:
X₁ and X₃ are independently NR¹, O, S, or PR²;
X₂ is N or P;
L is

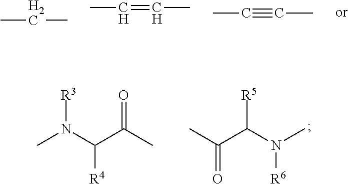

n is 0, 1, 2, 3, or 4;
R is H, CH₃, alkyl chain (linear or branched, number of carbons can be 1-4), cycloalkyl (ring size can be 3-7), —OR⁷, NH₂, NHR⁸, NR⁹R¹⁰, SH, SR¹¹, PH₂, PHR¹², PR¹³R¹⁴, halo, CN, NO₂, COOH, COOR¹⁵, CHO, COR¹⁶, CONH₂, CONHR¹⁷, CONR¹⁸R¹⁹, SOR²⁰, SO₂R²¹, SO₃R²², or chelator core;
Y₁-Y₁₇ are independently CH, CR²³ (wherein R²³ can be alkyl, alkenyl, alkynyl), —OH, —OR²⁴, NH₂, NHR²⁵, NR²⁶R²⁷, SH, SR²⁸, PH₂, PHR²⁹, PR³⁰R³¹, halo, BR³², BR³³R³⁴, CN, NO₂, COOH, COOR³⁴, CHO, COR³⁶, CONH₂, CONHR³⁷, CONR³⁸R³⁹, SOR⁴⁰, SO₂R⁴¹, SO₃R⁴², chelator core, CO, COO, COS, CONR⁴³, N, NH, NR⁴⁴, NO, O, S, SO, SO₂, BR⁴⁵, or BR⁴⁶R⁴⁷; and
R¹-R⁴⁷ are independently H, halo, C₁-C₁₂ linear alkyl, C₂-C₁₂ linear alkenyl, C₂-C₁₂ linear alkynyl, C₃-C₁₂ branched chain alkyl, C₃-C₁₂ branched chain alkenyl, C₃-C₁₂ branched chain alkynyl and C₃-C₇ cycloalkyl, or (hetero)aryl.

In some embodiments, the ROS imaging agent is preferentially localized to the brain or central nervous system (CNS); and/or an imaging signal of the ROS imaging agent is driven by a byproduct mediated by precursors of ROS.

In some embodiments, the subject has or is suspected of having a neurological or preclinical neurological disease, disorder, or condition, such as amyotrophic lateral sclerosis (ALS), Parkinson's disease (PD), Alzheimer's disease (AD), muscular dystrophy, or multiple sclerosis.

In some embodiments, the subject has or is suspected of having an inflammatory disease and/or long-COVID syndrome.

In some embodiments, the method further comprises exposing the subject to an imaging modality, such as PET or CT.

In some embodiments, administering the composition comprising the ROS imaging agent results in: penetration of the ROS imaging agent into a membrane; oxidation of the ROS imaging agent by ROS; and trapping of the ROS imaging agent in a cell membrane and intracellular compartments.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1 is a schematic showing the role of oxidative imbalance in mediating functional impairment of neuronal cells in accordance with the present disclosure.

FIG. 2 is a schematic showing synthesis of noncarrier added (unlabeled) 12 and carrier added 18F-SLN 128 (13; PET ROS Tracer) and its oxidized version (16; SLN 137) in accordance with the present disclosure.

FIG. 3 is a bar graph showing evaluation of SLN128 for detection of reactive oxygen species in vitro in accordance with the present disclosure. Data are shown as mean fluorescence intensity (arbitrary units)±SD, where ****p<0.0001 values are determined by an unpaired student t-test. (Abbreviations: G: Glucose; GOX: Glucose oxidase; HRP: Horseradish peroxidase; X: Xanthine; XO: Xanthine oxidase; SOD: Superoxide Dismutase; CAT: Catalase. All values are normalized to fluorescence output of untreated SLN128 (control). Please note response to HRP induced hydroxyl radical in GGO system, its mitigation with CAT; and response to HX-XO (superoxide) and its intervention with SOD.

FIG. 4 includes images showing cellular accumulation of SLN128 (20 nM) in human glioblastoma (U87) cells using live-cell fluorescence imaging: intracellular localization in accordance with the present disclosure. SLN128 was incubated at 37° C. for 1 h with either mitotracker red/or mitoSOX Deep Red in human U87MG cells. Images were acquired using a 100×objective. Note SLN 128 maps both mitotracker and mitoSOX indicating its ability to serve as reporter probe for mitochondrial ROS.

FIG. 5 includes images showing cellular accumulation of SLN128 (20 nM) in human glioblastoma (U87) cells using live-cell fluorescence imaging in accordance with the present disclosure. SLN128 was incubated for 1 h either with media (control), LPS treated (1 μg/mL; 3 h) or 3-nitropropionic acid (3-NP; 10 μg/mL) human U87MG cells: Images were acquired using a 20×objective.

FIG. 6 is a bar graph showing cellular accumulation of SLN128 in either serum and glucose deprived- or LPS or 3 NP-treated U87 cells in either presence or absence of resveratrol (10 μM) in accordance with the present disclosure. Images were acquired using a 20×objective (all panels represent same magnification) in live human U87 cells. While U87 control cells were plated under normal conditions, both serum-and glucose-deprived (Starvation), and LPS/3-NP treated cells were allowed to recover in presence of resveratrol) for 24 h and treated with SLN-128(20 nM) for 60 min, cell uptake was evaluated via live-cell imaging, and quantified as described earlier. Corrected Total Cell Fluorescence (CTCF)=Integrated Density-(Area of selected cell X Mean fluorescence of background readings); (mean±SD), where **p<0.01 and *p<0.1 are determined by an unpaired student t-test.

FIG. 7 is a bar graph showing cellular uptake of 18F-SLN 128 in human glioblastoma U87 cells in accordance with the present disclosure. Shown is net 18F-SLN 128 uptake (fmol×(nM0)−1×(mg protein)−1) at 60 min, using a control buffer either in the absence or presence of nutrients (starvation) and LPS. Each bar represents the mean of 4 determinations; lines above and below the bar denote±SD., wherein **p<0.01 and *p<0.1 are determined by an unpaired student t-test.

FIG. 8 is a line graph showing biodistribution of 18F-SLN128 in normal BI6 mice in accordance with the present disclosure. Shown is uptake (5 min, 60 min, 2 h) in brain and other critical organs quantified in terms of percent injected dose per gram of the tissue (% ID/g).

FIG. 9A-FIG. 9D is an exemplary embodiment showing preclinical PET/CT imaging in accordance with the present disclosure. C57BI6 mice (n=4) were injected with either LPS or saline intraperitoneally. 24 h post treatments, 18F-SLN-128 (100 μCi) was injected via tail-vein into mice. Dynamic PET images were acquired from 0 min to 60 min post tail-vein injection of the radiotracer. PET/CT images as shown are a summation frames from 45-60 min. FIG. 9A is an image showing saline treated mice (control). FIG. 9B is an image showing LPS treated mice (5 μg/g, 24 h post treatment); FIG. 9C is a bar graph showing quantified uptake: % ID/cm3. FIG. 9D is a bar graph showing post imaging biodistribution: Brain Uptake (% ID/g). mean±SD, where **p<0.01 and *p<0.1 is determined by an unpaired student ttest) in brains of BI6 mice (n=4) 24 h post either LPS- or saline treatments. Of note, 18F-SLN-128 shows stable retention in brains of LPS treated mice compared with their saline-treated counterparts indicating its ability to track LPS-induced inflammation.

FIG. 10A-FIG. 10D is an exemplary embodiment showing preclinical PET/CT imaging in accordance with the present disclosure. C57BI6 mice (n=4) were injected with either 3-Nitropropionic acid (3-NP) or saline into striatum. 72 h following treatments, 18F-SLN-128 was injected via tail-vein into mice. FIG. 10A includes PET/CT images shown as a summation frames from 45-60 min. Dynamic PET images acquired from 0 min to 60 min post tail-vein injection. Left: Control; Right: 3-NP treated (1 mg/kg, 72 h post treatment). FIG. 10B is a bar graph showing uantified uptake (% ID/cm³) from PET imaging. FIG. 10C includes images of brains extracted from mice, fixed in PFA/sucrose; sectioned (40 μm); stained with Cresyl violet; Arrow indicates injection site. FIG. 10D includes images of brain sections incubated with anti-NeuN antibody (neuronal marker) visualized by secondary antibody conjugated to Alexafluor488 and monoclonal anti-IBA1(ionized calcium-binding adapter molecule; gial marker) antibody visualized by secondary antibody conjugated to Alexafluor 568. Note that 18FSLN-128 brain uptake maps with neurodegeneration and microglial activation in the striatum regions. Slight uptake of the radiotracer in the right hemisphere of the brain indicates ability of the radiotracer to image earlier stages oxidative stress not detectable through immunohistochemistry (which detects either dying/dead neurons/glial cells).

FIG. 11 includes images showing post PET/CT imaging in accordance with the present disclosure. SLN128 (100 μL; 1 mg/mL) was injected through tail-vein into 3NP-or saline treated mice, and brain tissue was processed as described in FIG. 10A-FIG. 10D. Arrows indicate that SLN128 maps markers for microglial and neuronal cells in brain sections of 3-NP treated BI6 mice compared to none in saline treated control sections ex vivo thus indicating molecular specificity.

FIG. 12A-FIG. 12B is an exemplary embodiment showing dynamic MR/PET brain imaging of normal rhesus monkey (10 Kg) following intravenous injection of 18F-SLN-128 (5.8 mCi) over 2 h in accordance with the present disclosure. FIG. 12A includes images showing summation of PET data over 10 min and co-registered with T2-weighted MR image of the monkey for anatomical reference. FIG. 12B is a line graph showing SUVs as a time-activity curve for the tracer influx and clearance from important regions of the brain. Note: Insignificant nonspecific retention in different regions normal monkey brain.

### DETAILED DESCRIPTION

The present disclosure is based, at least in part, on the discovery of a new generation of redox-sensitive imaging agents. As shown herein, heterocyclic, highly fluorescent small organic molecules were developed capable of detecting ROS within mitochondria of neuronal cells.

Using radioactive tracers to assay cellular metabolism is one of the most attractive aspects of PET/CT imaging. One of the first radiotracers, called F18-FDG, is a workhorse of PET imaging, and can preferentially localize tumors based on their elevated glucose uptake.

There's a need to non-invasively image biochemical processes to better understand disease pathogenesis as well as the activity of pharmacologic interventions. Deep tissue imaging modalities based on MRI do not possess the sensitivity for such applications. Molecular imaging with radiotracers offers advantage of enabling non-invasive, quantitative and repetitive analysis of the biochemical status of tissues and organs.

However, few radiotracers can determine the generation of reaction oxygen species (ROS) with much fidelity. A standard clinical tracer called 18F-GDF, while capable of detecting glucose retention via Glut-1/hexokinase-I-mediated uptake and entrapment, poorly correlates with oxidative imbalance in the assayed tissue.

Described herein is a composition of matter for a class of heterocyclic imaging agents (e.g., NeuroROS) that react to the presence of reactive oxygen species, get trapped within the intracellular compartment, and output a signal that can be detected via PET/CT scanners. Therefore, these imaging agents offer a non-invasive, in vivo method to image ROS-generating cells or tissues. These compounds pass through cell membranes, but are preferentially localized to the brain after being injected intravenously. The NeuroROS tracers are extremely sensitive to generation of ROS (e.g. superoxide). Evolution of ROS immediately oxidizes the imaging agent and causes it to become trapped within the membrane compartment wherein the ROS-mediated oxidation occurred. The attached F18 radionuclide then outputs a signal to the PET/CT scanner, thus allowing measurement of quantitative accumulation of the NeuroROS agent based on the intensity of the radioactive signal.

The imaging agents described herein address the dearth of ROS-specific radiotracers. These imaging agents are preferentially localized to the CNS, but also have a relatively wide systemic distribution. ROS generation is understood to be a key step in the pathogenesis of Alzheimer's disease as well as a host of other neurodegenerative diseases. There's also evidence that elevated ROS generation precedes the accumulation of beta-amyloid as well tau. Thus, according to some embodiments disclosed herein, a non-invasive means to assay abnormal ROS generation is an early diagnosis of neurodegenerative diseases as well as providing a means to study pharmacologic intervention of this pathway on neurological outcomes.

Scientifically, the imaging agents described herein provide many opportunities to study the effects of ROS in different tissues, beyond just the brain. They can be coupled to the study of the effects of drugs, diets/lifestyle habits, as well as genetics on the generation of ROS. ROS studies have been very difficult to do in vivo due to the lack of effective tracers.

Endogenous ROS generation tends to occur earlier than more serious physiological signals such as inflammation, apoptosis/necrosis, or edema. The agents may therefore be an early readout of neurologic health.

Oxidative imbalance mediates pathogenesis of several neurodegenerative diseases including amyotrophic lateral sclerosis (ALS), Parkinson's disease (PD), and Alzheimer's disease (AD) and is shown to induce mitochondrial and synaptic dysfunction in neurons. However, noninvasive imaging tools to investigate role of oxidative imbalance in vivo have been lacking and continues to be an unmet need.

As described herein, a new generation of redox-sensitive molecular PET imaging probes (e.g., 18F-SLN-128) were developed through a rational design to address this critical gap in armamentarium of diagnostic agents. The probe penetrates neuronal cells, gets oxidized upon encountering oxidants, and trapped within cells to report on mitochondrial function.

Using live-cell fluorescence imaging analysis, herein is demonstrated the ability of molecular imaging probes to detect LPS- and 3-nitropropionic acid (3-NP)-induced oxidative imbalance within mitochondria of the human glioblastoma U87 cells. Moreover, in a model of LPS induced systemic inflammation of mouse brain, dynamic PET/CT scans revealed a 2-fold higher 18F-SLN128 uptake and retention in LPS-treated brains relative to uninjured saline-treated cohorts.

Furthermore, studies using a stereotaxic injection of 3-NP, a mitochondrial toxin into striatum demonstrates 2-fold higher retention of the radiotracer in brains of 3-NP treated mice compared with their saline treated counterparts. These data correlate with post-imaging quantitative biodistribution studies and immunohistochemical correlations thus providing evidence for microglial cell activation and neurodegeneration. Finally, dynamic PET/MR scan indicates ability of 18F-SLN-128 to penetrate brain (SUV=3.5) in a nontargeted rhesus monkey following intravenous injection of the radiotracer.

The probes described herein can enable monitoring oxidative-imbalance mediated pathogenesis in Parkinson's disease, muscular dystrophy, multiple sclerosis, amyotrophic lateral sclerosis, and chronic inflammation, thus extending benefits of this PET tracer well-beyond immediate utility in Alzheimer's disease and ADRDs. Additionally, long-COVID patients have shown persistent neurological deficits causing disabilities in the affected individuals. Underlying mechanisms in neurological syndrome remain unclear and may be influenced by factors such as site of the viral load, the differential immune response, neurodegenerative changes, and inflammation, however, mitochondrial functional impairment mediates pathogenesis of neuro-COVID in long-COVID syndrome. Therefore, in some embodiments these new molecular imaging agents are beneficial in understanding those mechanistic linkages thus extending benefits of noninvasive tools well beyond ADRDs.

### ROS Imaging Agents

Examples of ROS imaging agents are described herein. ROS imaging agents can be, for example, of any one of Formulas (I-VI)ₐ and (I-VII)_{b}:

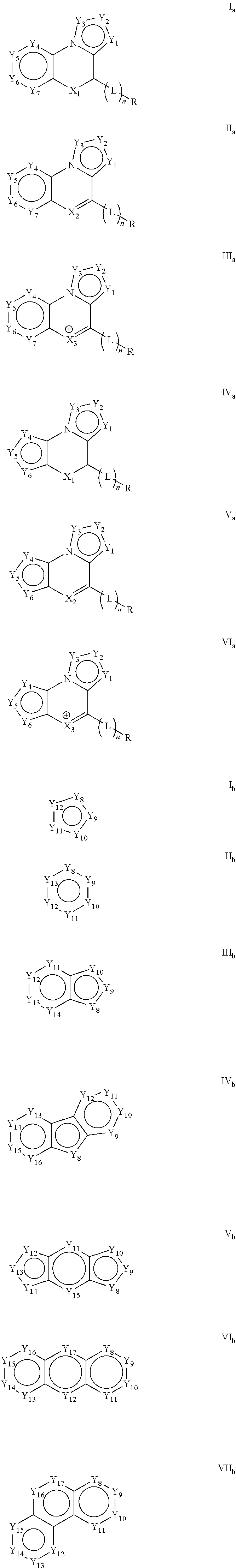

or a pharmaceutically acceptable salt, solvate, polymorph, tautomer, prodrug, analog, or stereoisomer thereof or optionally substituted analog thereof,
wherein
X₁ and X₃ may independently be NR¹, O, S, or PR²;
X₂ may be N or P;
L may be

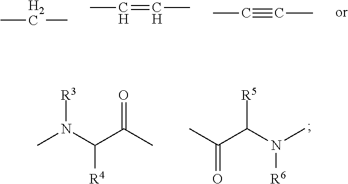

n may be 0, 1, 2, 3, or 4;
R may be H, CH₃, alkyl chain (linear or branched, number of carbons can be 1-4), cycloalkyl (ring size can be 3-7), —OR⁷, NH₂, NHR⁸, NR⁹R¹⁰, SH, SR¹¹, PH₂, PHR¹², PR¹³R¹⁴, halo, CN, NO₂, COOH, COOR¹⁵, CHO, COR¹⁶, CONH₂, CONHR¹⁷, CONR¹⁸R¹⁹, SOR²⁰, SO₂R²¹, SO₃R²², or chelator core;
Y₁-Y₁₇ may independently be CH, CR²³ (wherein R²³ can be alkyl, alkenyl, alkynyl), —OH, —OR²⁴, NH₂, NHR²⁵, NR²⁶R²⁷, SH, SR²⁸, PH₂, PHR²⁹, PR³⁰R³¹, halo, BR³², BR³³R³⁴, CN, NO₂, COOH, COOR³⁴, CHO, COR³⁶, CONH₂, CONHR³⁷, CONR³⁸R³⁹, SOR⁴⁰, SO₂R⁴¹, SO₃R⁴², chelator core, CO, COO, COS, CONR⁴³, N, NH, NR⁴⁴, NO, O, S, SO, SO₂, BR⁴⁵, or BR⁴⁶R⁴⁷.

R¹-R⁴⁷ may independently be H, halo, C₁-C₁₂ linear alkyl, C₂-C₁₂ linear alkenyl, C₂-C₁₂ linear alkynyl, C₃-C₁₂ branched chain alkyl, C₃-C₁₂ branched chain alkenyl, C₃-C₁₂ branched chain alkynyl and C3-C7 cycloalkyl, or (hetero)aryl.

The composition of claim 1, wherein the chelator core is NOTA, NODA, DOTA, DTPA, or Triglycine.

In some embodiments, H may be T or ³H.

In some embodiments, the halo may be Cl, F, Br, I, ¹⁸F, ⁷⁵Br, ⁷⁶Br, ⁷⁷Br, ¹²³I, ¹²⁴I, ¹²⁵I or ¹³¹I.

In some embodiments, the ROS imaging agent comprises a chelator core for chelation of a metal radionucle.

For example, the metal radionuclide may be an ion of gallium-67 (⁶⁷Ga), gallium-68 (⁶⁸Ga), an unlabeled gallium, or a paramagnetic metal which includes an ion of ⁶⁷Ga, an ion of ⁶⁸Ga, an ion of an unlabeled gallium, -indium-111 (¹¹¹In), -iron-52 (⁵²Fe), iron-59 (⁵⁹Fe), -copper-62 (⁶²Cu), -copper-64 (⁶⁴Cu), -thallium-201 (²⁰¹Tl) -technetium-99m (^{99m}Tc), -technetium-94m (^{94m}Tc), -rhenium-188 (¹⁸⁸Re), -rubidium-82 (⁸²Rb), -strontium-92 (⁹²Sr), -yttrium-86 (⁸⁶Y) or yttrium-90 (⁹⁰Y), -zirconium-86 (⁸⁶Zr) or -zirconium-89 (⁸⁹Zr), -aluminium fluoride-18 (Al¹⁸F), a paramagnetic metal ion, a transition metal (e.g., iron, manganese, or cobalt), or a lanthanide metal ion (such as gadolinium).

In some embodiments, the ROS imaging agent is:

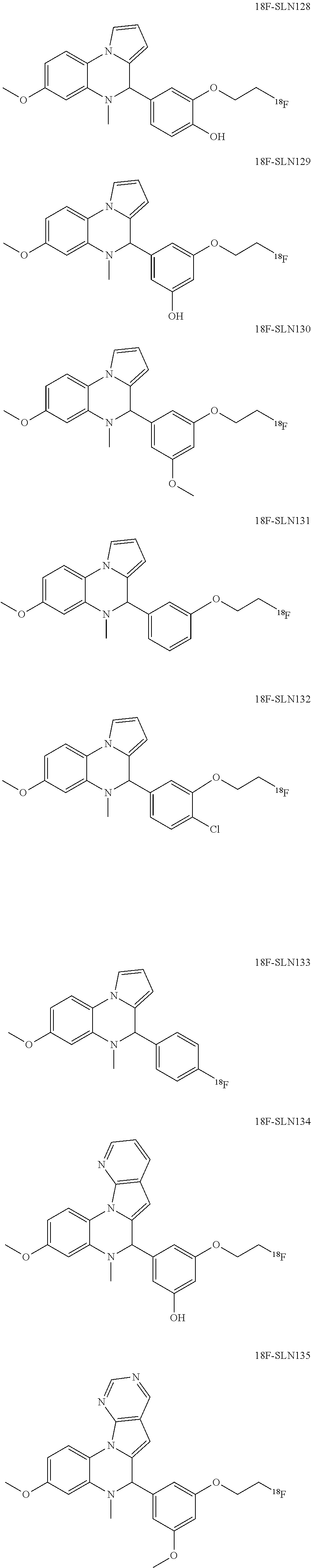

The formulas, analogs, and R groups can be optionally substituted or functionalized with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; and aryl comprising a phenyl; heteroaryl containing from 1 to 4 N, O, or S atoms; unsubstituted phenyl ring; substituted phenyl ring; unsubstituted heterocyclyl; and substituted heterocyclyl, wherein the unsubstituted phenyl ring or substituted phenyl ring can be optionally substituted with one or more groups independently selected from the group consisting of hydroxyl; C₁₋₁₀alkyl hydroxyl; amine; C₁₋₁₀carboxyl; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; aryl comprising a phenyl; and heteroaryl containing from 1 to 4 N, O, or S atoms; and the unsubstituted heterocyclyl or substituted heterocyclyl can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; heterocyclyl; straight chain or branched C₁₋₁₀alkyl amine; heterocyclic amine; and aryl comprising a phenyl; and heteroaryl containing from 1 to 4 N, O, or S atoms. Any of the above can be further optionally substituted.

The term “imine” or “imino”, as used herein, unless otherwise indicated, can include a functional group or chemical compound containing a carbon-nitrogen double bond. The expression “imino compound”, as used herein, unless otherwise indicated, refers to a compound that includes an “imine” or an “imino” group as defined herein. The “imine” or “imino” group can be optionally substituted.

The term “hydroxyl”, as used herein, unless otherwise indicated, can include —OH. The “hydroxyl” can be optionally substituted.

The terms “halogen” and “halo”, as used herein, unless otherwise indicated, include a chlorine, chloro, Cl; fluorine, fluoro, F; bromine, bromo, Br; or iodine, iodo, or I.

The term “acetamide”, as used herein, is an organic compound with the formula CH₃CONH₂. The “acetamide” can be optionally substituted.

The term “aryl”, as used herein, unless otherwise indicated, include a carbocyclic aromatic group. Examples of aryl groups include, but are not limited to, phenyl, benzyl, naphthyl, or anthracenyl. The “aryl” can be optionally substituted.

The terms “amine” and “amino”, as used herein, unless otherwise indicated, include a functional group that contains a nitrogen atom with a lone pair of electrons and wherein one or more hydrogen atoms have been replaced by a substituent such as, but not limited to, an alkyl group or an aryl group. The “amine” or “amino” group can be optionally substituted.

The term “alkyl”, as used herein, unless otherwise indicated, can include saturated monovalent hydrocarbon radicals having straight or branched moieties, such as but not limited to, methyl, ethyl, propyl, butyl, pentyl, hexyl, octyl groups, etc. Representative straight-chain lower alkyl groups include, but are not limited to, -methyl, -ethyl, -n-propyl, -n-butyl, -n-pentyl, -n-hexyl, -n-heptyl and -n-octyl; while branched lower alkyl groups include, but are not limited to, -isopropyl, -sec-butyl, -isobutyl, -tert-butyl, -isopentyl, 2-methylbutyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, unsaturated C₁₋₁₀ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, or -3-methyl-1 butynyl. An alkyl can be saturated, partially saturated, or unsaturated. The “alkyl” can be optionally substituted.

The term “carboxyl”, as used herein, unless otherwise indicated, can include a functional group consisting of a carbon atom double bonded to an oxygen atom and single bonded to a hydroxyl group (—COOH). The “carboxyl” can be optionally substituted.

The term “carbonyl”, as used herein, unless otherwise indicated, can include a functional group consisting of a carbon atom double-bonded to an oxygen atom (C═O). The “carbonyl” can be optionally substituted.

The term “alkenyl”, as used herein, unless otherwise indicated, can include alkyl moieties having at least one carbon-carbon double bond wherein alkyl is as defined above and including E and Z isomers of said alkenyl moiety. An alkenyl can be partially saturated or unsaturated. The “alkenyl” can be optionally substituted.

The term “alkynyl”, as used herein, unless otherwise indicated, can include alkyl moieties having at least one carbon-carbon triple bond wherein alkyl is as defined above. An alkynyl can be partially saturated or unsaturated. The “alkynyl” can be optionally substituted.

The term “acyl”, as used herein, unless otherwise indicated, can include a functional group derived from an aliphatic carboxylic acid, by removal of the hydroxyl (—OH) group. The “acyl” can be optionally substituted.

The term “alkoxyl”, as used herein, unless otherwise indicated, can include O-alkyl groups wherein alkyl is as defined above and O represents oxygen. Representative alkoxyl groups include, but are not limited to, —O-methyl, —O-ethyl, —O-n-propyl, —O-n-butyl, —O-n-pentyl, —O-n-hexyl, —O-n-heptyl, —O-n-octyl, —O-isopropyl, —O-sec-butyl, —O-isobutyl, —O-tert-butyl, —O-isopentyl, —O-2-methylbutyl, —O-2-methylpentyl, —O-3-methylpentyl, —O-2,2-dimethylbutyl, —O-2,3-dimethylbutyl, —O-2,2-dimethylpentyl, —O-2,3-dimethylpentyl, —O-3,3-dimethylpentyl, —O-2,3,4-trimethylpentyl, —O-3-methylhexyl, —O-2,2-dimethylhexyl, —O-2,4-dimethylhexyl, —O-2,5-dimethylhexyl, —O-3,5-dimethylhexyl, —O-2,4dimethylpentyl, —O-2-methylheptyl, —O-3-methylheptyl, —O-vinyl, —O-allyl, —O-1-butenyl, —O-2-butenyl, —O-isobutylenyl, —O-1-pentenyl, —O-2-pentenyl, —O-3-methyl-1-butenyl, —O-2-methyl-2-butenyl, —O-2,3-dimethyl-2-butenyl, —O-1-hexyl, —O-2-hexyl, —O-3-hexyl, —O-acetylenyl, —O-propynyl, —O-1-butynyl, —O-2-butynyl, —O-1-pentynyl, —O-2-pentynyl and —O-3-methyl-1-butynyl, —O-cyclopropyl, —O-cyclobutyl, —O-cyclopentyl, —O-cyclohexyl, —O-cycloheptyl, —O-cyclooctyl, —O-cyclononyl and —O-cyclodecyl, —O—CH₂-cyclopropyl, —O—CH₂-cyclobutyl, —O—CH₂-cyclopentyl, —O—CH₂-cyclohexyl, —O—CH₂-cycloheptyl, —O—CH₂-cyclooctyl, —O—CH₂-cyclononyl, —O—CH₂-cyclodecyl, —O—(CH₂)₂-cyclopropyl, —O—(CH₂)₂-cyclobutyl, —O—(CH₂)₂-cyclopentyl, —O—(CH₂)₂-cyclohexyl, —O—(CH₂)₂-cycloheptyl, —O—(CH₂)₂-cyclooctyl, —O—(CH₂)₂-cyclononyl, or —O—(CH₂)₂-cyclodecyl. An alkoxyl can be saturated, partially saturated, or unsaturated. The “alkoxyl” can be optionally substituted.

The term “cycloalkyl”, as used herein, unless otherwise indicated, can include an aromatic, a non-aromatic, saturated, partially saturated, or unsaturated, monocyclic or fused, spiro or unfused bicyclic or tricyclic hydrocarbon referred to herein containing a total of from 1 to 10 carbon atoms (e.g., 1 or 2 carbon atoms if there are other heteroatoms in the ring), preferably 3 to 8 ring carbon atoms. Examples of cycloalkyls include, but are not limited to, C₃₋₁₀ cycloalkyl groups include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. The term “cycloalkyl” also can include -lower alkyl-cycloalkyl, wherein lower alkyl and cycloalkyl are as defined herein. Examples of -lower alkyl-cycloalkyl groups include, but are not limited to, —CH₂-cyclopropyl, —CH₂-cyclobutyl, —CH₂-cyclopentyl, —CH₂-cyclopentadienyl, —CH₂-cyclohexyl, —CH₂-cycloheptyl, or —CH₂-cyclooctyl. The “cycloalkyl” can be optionally substituted. A “cycloheteroalkyl”, as used herein, unless otherwise indicated, can include any of the above with a carbon substituted with a heteroatom (e.g., O, S, N).

The term “heterocyclic” or “heteroaryl”, as used herein, unless otherwise indicated, can include an aromatic or non-aromatic cycloalkyl in which one to four of the ring carbon atoms are independently replaced with a heteroatom from the group consisting of O, S, and N. Representative examples of a heterocycle include, but are not limited to, benzofuranyl, benzothiophene, indolyl, benzopyrazolyl, coumarinyl, isoquinolinyl, pyrrolyl, pyrrolidinyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl, (1,4)-dioxane, (1,3)-dioxolane, 4,5-dihydro-1H-imidazolyl, or tetrazolyl. Heterocycles can be substituted or unsubstituted. Heterocycles can also be bonded at any ring atom (i.e., at any carbon atom or heteroatom of the heterocyclic ring). A heterocyclic can be saturated, partially saturated, or unsaturated. The “hetreocyclic” can be optionally substituted.

The term “indole”, as used herein, is an aromatic heterocyclic organic compound with formula C₈H₇N. It has a bicyclic structure, consisting of a six-membered benzene ring fused to a five-membered nitrogen-containing pyrrole ring. The “indole” can be optionally substituted.

The term “cyano”, as used herein, unless otherwise indicated, can include a —CN group. The “cyano” can be optionally substituted.

The term “alcohol”, as used herein, unless otherwise indicated, can include a compound in which the hydroxyl functional group (—OH) is bound to a carbon atom. In particular, this carbon center should be saturated, having single bonds to three other atoms. The “alcohol” can be optionally substituted.

The term “solvate” is intended to mean a solvate form of a specified compound that retains the effectiveness of such compound. Examples of solvates include compounds of the invention in combination with, for example, water, isopropanol, ethanol, methanol, dimethylsulfoxide (DMSO), ethyl acetate, acetic acid, or ethanolamine.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “μM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “HPLC”, as used herein, is intended to mean high performance liquid chromatograph. The term “RT”, as used herein, is intended to mean room temperature. The term “e.g.”, as used herein, is intended to mean example. The term “N/A”, as used herein, is intended to mean not tested.

As used herein, the expression “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion, or another counterion. The counterion may be any organic or inorganic moiety that stabilizes the charge on the parent compound. Furthermore, a pharmaceutically acceptable salt may have more than one charged atom in its structure. In instances where multiple charged atoms are part of the pharmaceutically acceptable salt, the pharmaceutically acceptable salt can have multiple counterions. Hence, a pharmaceutically acceptable salt can have one or more charged atoms and/or one or more counterion. As used herein, the expression “pharmaceutically acceptable solvate” refers to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine. As used herein, the expression “pharmaceutically acceptable hydrate” refers to a compound of the invention, or a salt thereof, that further can include a stoichiometric or non-stoichiometric amount of water bound by non-covalent intermolecular forces.

### Neurological Disease, Disorder, or Condition

The present disclosure provides for methods and compositions to detect reactive oxygen species (ROS) in a subject having or suspected of having neurological diseases, disorders, or conditions. For example, a neurological disease, disorder, or condition can be Abulia; Agraphia; Alcoholism; Alexia; Alien hand syndrome; Allan-Herndon-Dudley syndrome; Alternating hemiplegia of childhood; Alzheimer's disease; Amaurosis fugax; Amnesia; Amyotrophic lateral sclerosis (ALS); Aneurysm; Angelman syndrome; Anosognosia; Aphasia; Apraxia; Arachnoiditis; Arnold-Chiari malformation; Asomatognosia; Asperger syndrome; Ataxia; Attention deficit hyperactivity disorder; ATR-16 syndrome; Auditory processing disorder; Autism spectrum; Behcets disease; Bipolar disorder; Bell's palsy; Brachial plexus injury; Brain damage; Brain injury; Brain tumor; Brody myopathy; Canavan disease; Capgras delusion; Carpal tunnel syndrome; Causalgia; Central pain syndrome; Central pontine myelinolysis; Centronuclear myopathy; Cephalic disorder; Cerebral aneurysm; Cerebral arteriosclerosis; Cerebral atrophy; Cerebral autosomal dominant arteriopathy with subcortical infarcts and leukoencephalopathy (CADASIL); Cerebral dysgenesis-neuropathy-ichthyosis-keratoderma syndrome (CEDNIK syndrome); Cerebral gigantism; Cerebral palsy; Cerebral vasculitis; Cervical spinal stenosis; Charcot-Marie-Tooth disease; Chiari malformation; Chorea; Chronic fatigue syndrome; Chronic inflammatory demyelinating polyneuropathy (CIDP); Chronic pain; Cockayne syndrome; Coffin-Lowry syndrome; Coma; Complex regional pain syndrome; Compression neuropathy; Congenital facial diplegia; Corticobasal degeneration; Cranial arteritis; Craniosynostosis; Creutzfeldt-Jakob disease; Cumulative trauma disorders; Cushing's syndrome; Cyclothymic disorder; Cyclic Vomiting Syndrome (CVS); Cytomegalic inclusion body disease (CIBD); Cytomegalovirus Infection; Dandy-Walker syndrome; Dawson disease; De Morsier's syndrome; Dejerine-Klumpke palsy; Dejerine-Sottas disease; Delayed sleep phase syndrome; Dementia; Dermatomyositis; Developmental coordination disorder; Diabetic neuropathy; Diffuse sclerosis; Diplopia; Disorders of consciousness; Down syndrome; Dravet syndrome; Duchenne muscular dystrophy; Dysarthria; Dysautonomia; Dyscalculia; Dysgraphia; Dyskinesia; Dyslexia; Dystonia; Empty sella syndrome; Encephalitis; Encephalocele; Encephalotrigeminal angiomatosis; Encopresis; Enuresis; Epilepsy; Epilepsy-intellectual disability in females; Erb's palsy; Erythromelalgia; Essential tremor; Exploding head syndrome; Fabry's disease; Fahr's syndrome; Fainting; Familial spastic paralysis; Febrile seizures; Fisher syndrome; Friedreich's ataxia; Fibromyalgia; Foville's syndrome; Fetal alcohol syndrome; Fragile X syndrome; Fragile X-associated tremor/ataxia syndrome (FXTAS); Gaucher's disease; Generalized epilepsy with febrile seizures plus; Gerstmann's syndrome; Giant cell arteritis; Giant cell inclusion disease; Globoid Cell Leukodystrophy; Gray matter heterotopia; Guillain-Barré syndrome; Generalized anxiety disorder; HTLV-1 associated myelopathy; Hallervorden-Spatz syndrome; Head injury; Headache; Hemifacial Spasm; Hereditary Spastic Paraplegia; Heredopathia atactica polyneuritiformis; Herpes zoster oticus; Herpes zoster; Hirayama syndrome; Hirschsprung's disease; Holmes-Adie syndrome; Holoprosencephaly; Huntington's disease; Hydranencephaly; Hydrocephalus; Hypercortisolism; Hypoxia; Immune-Mediated encephalomyelitis; Inclusion body myositis; Incontinentia pigmenti; Infantile Refsum disease; Infantile spasms; Inflammatory myopathy; Intracranial cyst; Intracranial hypertension; Isodicentric 15; Joubert syndrome; Karak syndrome; Kearns-Sayre syndrome; Kinsbourne syndrome; Kleine-Levin syndrome; Klippel Feil syndrome; Krabbe disease; Kufor-Rakeb syndrome; Lafora disease; Lambert-Eaton myasthenic syndrome; Landau-Kleffner syndrome; Lateral medullary (Wallenberg) syndrome; Learning disabilities; Leigh's disease; Lennox-Gastaut syndrome; Lesch-Nyhan syndrome; Leukodystrophy; Leukoencephalopathy with vanishing white matter; Lewy body dementia; Lissencephaly; Locked-in syndrome; Lou Gehrig's disease (See amyotrophic lateral sclerosis); Lumbar disc disease; Lumbar spinal stenosis; Lyme disease—Neurological Sequelae; Machado-Joseph disease (Spinocerebellar ataxia type 3); Macrencephaly; Macropsia; Mal de debarquement; Megalencephalic leukoencephalopathy with subcortical cysts; Megalencephaly; Melkersson-Rosenthal syndrome; Menieres disease; Meningitis; Menkes disease; Metachromatic leukodystrophy; Microcephaly; Micropsia; Migraine; Miller Fisher syndrome; Mini-stroke (transient ischemic attack); Misophonia; Mitochondrial myopathy; Mobius syndrome; Monomelic amyotrophy; Morvan syndrome; Motor Neuron Disease—see amyotrophic lateral sclerosis; Motor skills disorder; Moyamoya disease; Mucopolysaccharidoses; Multi-infarct dementia; Multifocal motor neuropathy; Multiple sclerosis; Multiple system atrophy; Muscular dystrophy; Myalgic encephalomyelitis; Myasthenia gravis; Myelinoclastic diffuse sclerosis; Myoclonic Encephalopathy of infants; Myoclonus; Myopathy; Myotubular myopathy; Myotonia congenita; Narcolepsy; Neuro-Behçet's disease; Neurofibromatosis; Neuroleptic malignant syndrome; Neurological manifestations of AIDS; Neurological sequelae of lupus; Neuromyotonia; Neuronal ceroid lipofuscinosis; Neuronal migration disorders; Neuropathy; Neurosis; Niemann-Pick disease; Non-24-hour sleep-wake disorder; Nonverbal learning disorder; O'Sullivan-McLeod syndrome; Occipital Neuralgia; Occult Spinal Dysraphism Sequence; Ohtahara syndrome; Olivopontocerebellar atrophy; Opsoclonus myoclonus syndrome; Optic neuritis; Orthostatic Hypotension; Otosclerosis; Overuse syndrome; Palinopsia; Paresthesia; Parkinson's disease; Paramyotonia congenita; Paraneoplastic diseases; Paroxysmal attacks; Parry-Romberg syndrome; PANDAS; Pelizaeus-Merzbacher disease; Periodic paralyses; Peripheral neuropathy; Pervasive developmental disorders; Phantom limb/Phantom pain; Photic sneeze reflex; Phytanic acid storage disease; Pick's disease; Pinched nerve; Pituitary tumors; PMG; Polyneuropathy; Polio; Polymicrogyria; Polymyositis; Porencephaly; Post-polio syndrome; Postherpetic neuralgia (PHN); Postural hypotension; Prader-Willi syndrome; Primary lateral sclerosis; Prion diseases; Progressive hemifacial atrophy; Progressive multifocal leukoencephalopathy; Progressive supranuclear palsy; Prosopagnosia; Pseudotumor cerebri; Quadrantanopia; Quadriplegia; Rabies; Radiculopathy; Ramsay Hunt syndrome type I; Ramsay Hunt syndrome type II; Ramsay Hunt syndrome type III—see Ramsay-Hunt syndrome; Rasmussen encephalitis; Reflex neurovascular dystrophy; Refsum disease; REM sleep behavior disorder; Repetitive stress injury; Restless legs syndrome; Retrovirus-associated myelopathy; Rett syndrome; Reye's syndrome; Rhythmic Movement Disorder; Romberg syndrome; Saint Vitus dance; Sandhoff disease; Schilder's disease (two distinct conditions); Schizencephaly; Sensory processing disorder; Septo-optic dysplasia; Shaken baby syndrome; Shingles; Shy-Drager syndrome; Sjögren's syndrome; Sleep apnea; Sleeping sickness; Snatiation; Sotos syndrome; Spasticity; Spina bifida; Spinal cord injury; Spinal cord tumors; Spinal muscular atrophy; Spinal and bulbar muscular atrophy; Spinocerebellar ataxia; Split-brain; Steele-Richardson-Olszewski syndrome; Stiff-person syndrome; Stroke; Sturge-Weber syndrome; Stuttering; Subacute sclerosing panencephalitis; Subcortical arteriosclerotic encephalopathy; Superficial siderosis; Sydenham's chorea; Syncope; Synesthesia; Syringomyelia; Tarsal tunnel syndrome; Tardive dyskinesia; Tardive dysphrenia; Tarlov cyst; Tay-Sachs disease; Temporal arteritis; Temporal lobe epilepsy; Tetanus; Tethered spinal cord syndrome; Thomsen disease; Thoracic outlet syndrome; Tic Douloureux; Todd's paralysis; Tourette syndrome; Toxic encephalopathy; Transient ischemic attack; Transmissible spongiform encephalopathies; Transverse myelitis; Traumatic brain injury; Tremor; Trichotillomania; Trigeminal neuralgia; Tropical spastic paraparesis; Trypanosomiasis; Tuberous sclerosis; 22q13 deletion syndrome; Unverricht-Lundborg disease; Vestibular schwannoma (Acoustic neuroma); Von Hippel-Lindau disease (VHL); Viliuisk Encephalomyelitis (VE); Wallenberg's syndrome; West syndrome; Whiplash; Williams syndrome; Wilson's disease; Y-Linked Hearing Impairment; or Zellweger syndrome.

### Formulation

The agents and compositions described herein can be formulated by any conventional manner using one or more pharmaceutically acceptable carriers or excipients as described in, for example, Remington's Pharmaceutical Sciences (A.R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A.R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve taste of the product; or improve shelf life of the product.

### Screening

Also provided are screening methods.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 MW, or less than about 1000 MW, or less than about 800 MW) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example, ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character x log P of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character x log P of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of a compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to imaging agents or radiolabels. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject or sample, a wild-type subject or sample, or from populations thereof. A reference value can be used in place of a control or reference sample, which was previously obtained from a healthy subject or a group of healthy subjects or a wild-type subject or sample. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Novel Heterocyclic Molecules for Detecting ROS

This Example describes a novel template scaffold, comprising a heterocyclic, highly fluorescent small organic molecule capable of detecting ROS within mitochondria of neuronal cells.

### Introduction

Oxidative imbalance mediates pathogenesis of AD, PD, and other neurodegenerative diseases, and induces mitochondrial and synaptic dysfunction in neurons. For patients with mild cognitive impairment (MCI) due to AD, protein carbonyl modifications are present in cerebrospinal fluid (CSF). The hippocampi of patients with MCI show significant oxidative modifications in biological macromolecules. In addition, oxidative stress induces lipid bilayer modification thus resulting in increased levels of 4-hydroxy 2-nonenal in the CSF and brain of patients with AD. The elevated protein carbonyl content in brain regions enriched with senile plaques is also indicative of an association between oxidative damage and AD. Furthermore, the brains of patients with MCI and AD also have increased oxidative alterations, such as protein nitration and nucleic acid modifications. Fluorodeoxyglucose-positive emission tomography (FDG-PET) imaging analysis revealed suppressed brain glucose metabolism in MCI and AD pathogenesis, suggesting the involvement of mitochondria. Interestingly, such a reduction in the glucose metabolism poorly correlates with Aβ plaques or τ tangle pathology. Substantial evidence reveals that the loss of functional synapses marks an early event in AD pathogenesis. This synaptic loss correlates with memory impairment during the early stages of the disease, even before any loss of neurons becomes apparent. The spread of tau pathology across the brain regions is hypothesized to take place via a synaptic route. Thus, the pathology of dysfunctional synapses in AD could be linked to oxidative stress (OS) and inadequate quenching mechanisms of antioxidants. The synaptic dysfunction in AD is also associated with NMDA and AMPA receptor-mediated excitotoxicity and inefficient compensatory GABAergic brain circuitry. During early-stage AD and mild cognitive impairment (MCI), Aβ oligomers promote synaptic membrane degeneration leading to a deficit in the long-term potentiation, and subsequent loss of memory and learning. Thus, oxidative modifications, impaired glucose metabolism, and insufficient ATP production by mitochondria could contribute to subsequent membrane potential alterations, aberrant firing of action potentials, calcium imbalance, and deficits in neurotransmission. These cellular and functional abnormalities lead to structural modifications of synapses and dendrites resulting in learning and memory impairments, decreased executive functions and diminished reasoning in patients with MCI and AD.

These literature precedents unequivocally suggest that the markers of OS (advanced glycation end products, ROS, lipid peroxides, hydroxyl radical adducts with DNA bases, and carbonyls) increase in the brains of patients with AD and also in the experimental rodent models of AD. Furthermore, the accumulation of the classical AD biomarkers, such as Tau and Aβ proteins along with the mitochondrial dysfunction enhances oxidative stress in the brain. Importantly, ROS-induced structural and functional disruption of the cell membrane and other cellular components have been postulated as the primary drivers of the pathogenesis of cognitive decline and AD.

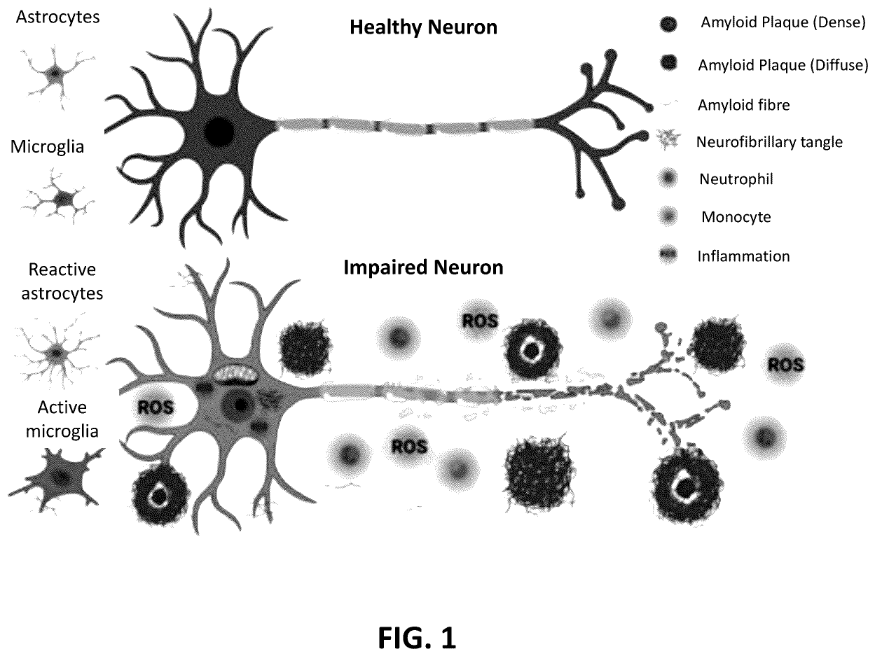

Clinical studies have also demonstrated increases in the levels of lipid peroxidation markers (F2-IsoPs, F4-NeuroPs, malondialdehyde-MDA, and protein-bound acrolein adduct) in the frontal and temporal lobes of AD brains. Of note, investigations have highlighted that these markers were significantly higher in the inferior parietal lobe and occipital area of patients with MCI compared with control subjects. These findings also correlate with the alterations in the antioxidative defenses in the brains of patients with AD. Importantly, postmortem investigations have revealed decreased GSH levels and increased GSSG levels in both patients with MCI and severe AD. These provocative findings indicate a direct correlation between the reduced GSH levels in the brain and a cognitive decline. Recent studies have generated evidence of the increased SOD activity and reduced GPx, CAT, and peroxiredoxin (Prx) activities in the superior temporal gyrus of the brains of patients with AD. Substantial preclinical evidence exists for an increase in the OS in the hippocampus and cortex of the brain during AD progression. For example, administration of Aβ1-42 peptide into rat forebrain induces oxidative stress in the hippocampus, whereas local infusion of oxidizing agents into the hippocampus of mice increased local Aβ42 levels in the interstitial fluid. These experimental data indicate that OS likely acts upstream in Aβ pathology. The levels of a lipid peroxidation marker (8,12-iso-iPF2α-VI) are significantly higher in the urine, plasma, and brain tissue samples of Tg2576 mice. Combined factors provide compelling evidence for role of oxidative imbalance in conjunction with misfolded proteins (Aβ and p-Tau) at the front and center of AD pathogenesis resulting in functional impairment of neurons (see e.g., FIG. 1), however, noninvasive imaging tools to investigate role of oxidative imbalance in vivo have been lacking and continues to be an unmet need.

### Significance

Conventional deep tissue tomographic imaging techniques, such as contrast-enhanced magnetic resonance imaging or CT, can highlight areas of edema associated with inflammation but lack the specificity to differentiate non-inflammatory edema. Molecular imaging with radiotracers offers advantage of enabling non-invasive, quantitative and repetitive analysis of the biochemical status of tissues and organs. To address this need, standard clinical [¹⁸F]FDG PET/CT lacks sensitivity and diagnostic robustness. Importantly, mechanism of [¹⁸F]FDG retention via Glut-1/hexokinase-I-mediated uptake and entrapment poorly correlates with oxidative imbalance. Therefore, among the molecular imaging community, there is a growing recognition of substantial unmet need for new radiotracers that can identify and spatially and temporally localize focal inflammation (based on the activation state of the innate immune system) in vivo. Among various insults, oxidative imbalance mediated modification of proteins, lipids, carbohydrates, and nucleic acids alters their stability and function. Furthermore, the endogenous oxidative imbalance has also been postulated to drive genomic instability, stimulate accumulation of misfolded proteins, and modification of numerous key processes, such as proteostasis, autophagy, and mitochondrial function. This latter phenomenon is particularly relevant in the brain: because of the prevalently oxidative metabolism needed to meet massive Gibbs energy requirements throughout life, thus neurons have a high rate of ROS production. Of note, numerous studies using postmortem tissue from individuals with preclinical AD, mild cognitive impairment, and AD show evidence of oxidative imbalance and peroxidated lipids. Whether oxidative imbalance is a cause or consequence of the disease remains an open-ended question thus intensely investigated in laboratories worldwide. Therefore, it is also likely that ROS production, Aβ-, and p-Tau depositions are intertwined with inflammation and neurodegeneration. It is also conceivable that elevated levels of oxidants in brain precede both Aβ, p-Tau depositions including other misfolded proteins (α-synuclein and TDP) mediating neurodegenerative disorders and could offer druggable targets at prodromal stages prior to clinical expression of the disease. For investigating these critical relationships, wherein elevated levels of ROS mediate pathogenesis of neurodegenerative diseases in general, and ADRDs in particular; unfortunately, currently there are no FDA approved molecularly specific and -sensitive PET tracers to interrogate the role of this important biomarker and quantify efficacy of therapeutic interventions in vivo. To address this unmet need in clinical diagnostic nuclear medicine, while exploring structure-activity relationship studies for designing PET tracers for imaging neuroinflammation, identified herein is a novel template scaffold, comprising a heterocyclic, highly fluorescent small organic molecule capable of detecting ROS within mitochondria of neuronal cells thus providing a highly versatile molecular imaging probe for noninvasive interrogation of impaired mitochondrial function within the brain. This template scaffold could also provide a platform for development of other molecular-specific and -sensitive molecular PET imaging tracers, which can be deployed for monitoring ROS-mediated pathogenesis in Parkinson's disease, muscular dystrophy, multiple sclerosis, amyotrophic lateral sclerosis, and chronic inflammation, thus extending benefits of this PET tracer well-beyond immediate utility in Alzheimer's disease and ADRDs. Finally, long-COVID patients have shown persistent neurological deficits causing disabilities in the affected individuals. Although underlying mechanisms in neurological syndrome remain unclear, and could be influenced by factors such as site of the viral load, the differential immune response, neurodegenerative changes, and inflammation. Given that mitochondrial ROS (mROS) is at front and center of these biochemical pathways, it is likely that mitochondrial functional impairment mediates pathogenesis of neuro-COVID in long-COVID syndrome. Therefore, molecular imaging agents capable of providing noninvasive readout of mROS could also be beneficial in understanding those mechanistic linkages thus extending benefits of noninvasive tools well beyond ADRDs.

### Innovation

For enabling investigation of mROS-induced functional impairment of neuron and glial cells, identified herein is a lead agent (¹⁸F-SLN-128) that demonstrates traits, desirable of imaging agents with translational potential, while also incorporated with ¹⁸F, a PET isotope of choice for medical PET imaging. The proposed project is both technically and conceptually novel. While technical novelty comes from the fact that molecular design for the probe involves structural features, which will allow comparative analysis of inherent fluorescence output correlations with PET data to enable tracking the site of inflammatory process with precision and quantification of the signal at the cellular level (neutrophils, macrophages, microglial cells) and their direct relevance to the disease staging. Following transfer of the technology from the bench to the bedside, the PET tracer could offer companion diagnostic for understanding pathogenesis of the disease at prodromal stages for managing patients with AD and ADRDs. Of note, molecular imaging probe belongs to an entirely new class of heterocyclic molecules, while demonstrating pharmacokinetic profile desirable in PET tracers worthy of clinical translation. The conceptual innovation of proposed project comes from a rational design of the redox sensitive molecular imaging probe, wherein the probe penetrates neuronal cells and gets oxidized upon encountering oxidants (superoxide and hydroxyl free radical) converts (in vivo) from a neutral molecule into a charged species. Thus, this strategic design takes advantage of favorable transmembrane gradients to allow trapping within mitochondria of neuronal and glial cells having elevated levels of ROS. Therefore, the strategic probe would result into enhanced retention in regions of high ROS and excretion (neutral form) from regions lacking ROS to afford high target/background ratios for imaging AD and ADRDs to allow stratification of therapeutic choices or modification of lifestyles. This project is feasible due to: a) the lead agent shows ability to monitor LPS-induced inflammation in the mice brains (microPET/CT imaging and Post Imaging biodistribution data); b) the lead agent tracks 3-nitropropionic acid (3-NP) induced ROS and neurodegeneration; and c) probe penetrates brains of NHPs and excretes to baseline signal. Finally, successful accomplishment of proposed aims could also deliver molecular imaging agent for management of ROS-mediated pathogenesis in Parkinson's disease, muscular dystrophy, multiple sclerosis, amyotrophic lateral sclerosis, stroke, traumatic brain injury, and chronic inflammation, thus extending benefits for functional imaging of mitochondrial function in vivo thus extending its outreach well-beyond immediate utility in Alzheimer's disease and ADRDs.

### Supporting Data

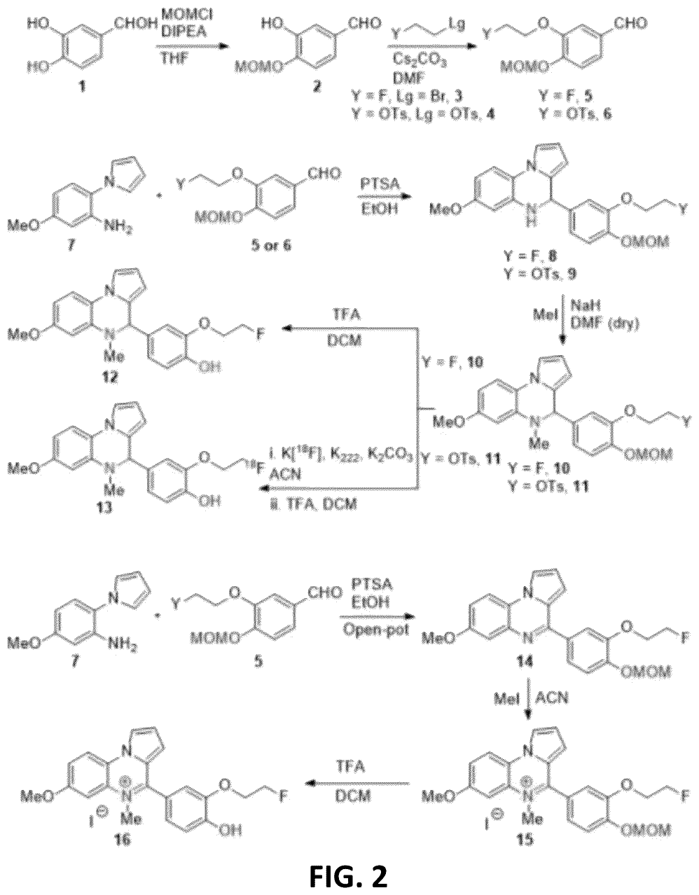

Oxidative imbalance and functional impairment of mitochondria may mediate pathogenesis of neurodegenerative diseases and mitochondria are among the prominent intracellular site for ROS generation. Thus, a 2nd generation redox-sensitive neuroROS tracer was designed (also identified as either as SLN 128 or 12 (noncarrier added) or ¹⁸F-13 (PET tracer); see e.g., FIG. 2) for the detection of total ROS within cells undergoing pro-oxidative stage. While the unlabeled/noncarrier counterpart of the probe 12 was synthesized and characterized through standard analytical methods (Proton NMR, proton-decoupled 13C-NMR, HRMS, Elemental analysis), the carrier added ¹⁸F-13 (Lab ID:18F-SLN128) was synthesized through a reaction between the tosylated precursor 11 and KF{18F}-/Kryptofix 222 and followed by deprotection of methoxymethyl ether 11 in situ with TFA as shown (see e.g., FIG. 2), purified on semipreparatory C-18 column, the desired fraction with a Rt=12 min was collected, concentrated, reconstituted in 5% ethanol/95%sterile saline and analyzed for purity on QC radio-HPLC (radiochemical yield: 15-20%; radiochemical purity >99%; specific activity:1400-1700 Ci/mmole) and identified by spiking with the noncarrier added/unlabeled counterpart. To obtain oxidized counterpart, 5-methoxy-2-(1H-pyrrol-1-yl)aniline (7) and 3-(2-fluoroethoxy)-4-(methoxymethoxy)benzaldehyde (5) were treated with PTSA in ethanol to obtain 14, which was treated with methyl iodide to yield 15, which upon treatment with TFA yielded oxidized counterpart 16 (Lab IB: SLN 137) and demonstrated a retention Rt=4.8 min under identical conditions. For analysis of metabolites, ¹⁸F-SLN-128 was incubated with either human serum or plasma at 37° C. as a function of time and analyzed on QC Radio-HPLC. 18F-SLN-128 (18F-13) remains non-metabolized in human serum upto 1 h to enable delayed imaging without competing metabolites.

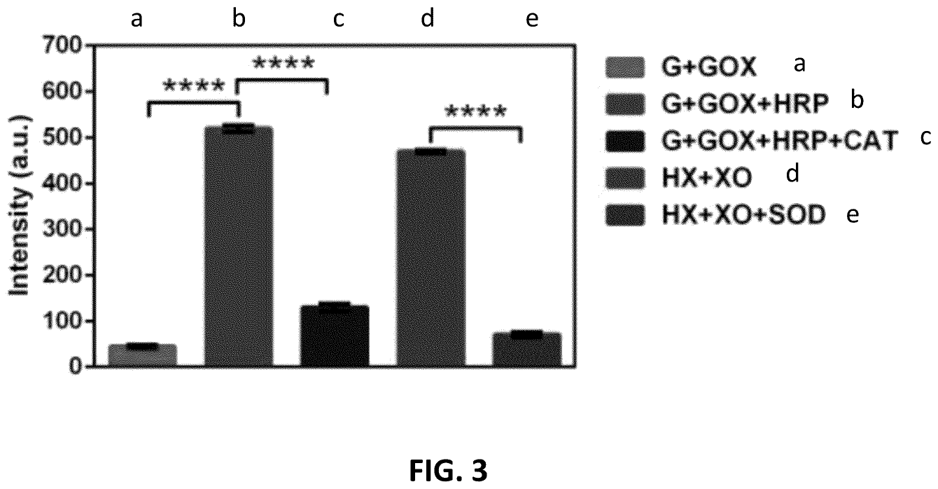

Of note, SLN128 (7), when solubilized in PBS with 1.0% ethanol, and is excited with a 402 nm laser, it exhibits a broad emission peak between 520-540 nm with a maximum of 530 nm, however, following its oxidation (15), its mean fluorescence intensity shows a 4-fold enhancement accompanied by a shift to 580 nm thus offering critical trait for its biochemical validation through live-cell fluorescence imaging. Of note, superoxide can originate from numerous sources in vivo, including the mitochondrial electron transport chain and NADPH oxidase(s), which are activated under conditions of inflammation, ischemia, and tissue injury. Superoxide has also been shown to regulate distinct signaling cascades with specific molecular targets. Further, the hydroxyl radical, the most potent oxidant, can also be produced by Fenton reaction involving iron(II) and hydrogen peroxide. Therefore, the ability to detect elevated ROS in patients could provide a better understanding of relationships between ROS, tissue injury, and disease staging including information on signaling cascade that promote inflammation. To measure ROS production, various methods that use colorimetry, chemiluminescence, and fluorescence have been deployed, While xanthine-xanthine oxidase (X-XO) and SIN-1 have been used for production of superoxide and nitric oxide (wherein the combination produces peroxynitrite), respectively, the glucose-glucose oxidase (G-GO) system is useful for production of hydrogen peroxide. Employing concentrations of SLN128 and G-GO, and X-XO in vitro conditions described previously, the ability of SLN-128 or 12 to detect different reactive oxygen species at 37° C. in PBS was evaluated under physiological conditions (pH 7.2) and normalized by background fluorescence output of untreated SLN128 as a control using a fluorescence plate reader assay (see e.g., FIG. 3). SLN128 showed the ability to detect superoxide generated through either xanthine oxidase metabolism of xanthine or by thermal decomposition of SIN-1 in the presence of CPTIO, an inhibitor of nitric oxide. Under both conditions, oxidation of the probe was neutralized by the addition of superoxide dismutase (SOD) (see e.g., FIG. 3). While SLN128 shows only a slight increase in fluorescence in G-GO system, it shows substantial 10-fold higher response to HRP in G-GO system, thus indicating its ability to detect hydroxyl radical. (see e.g., FIG. 3). Compared with ¹⁸F-ROStrace, which detects only superoxide, SLN128 shows capability to detect both superoxide and hydroxyl radical (high redox potential (2.7 V), the most lethal oxidant shown to modify biomolecules. Therefore, SLN 128 offers advantages over ¹⁸F-ROStrace for imaging pathogenesis of neurodegenerative diseases, including AD and PD.

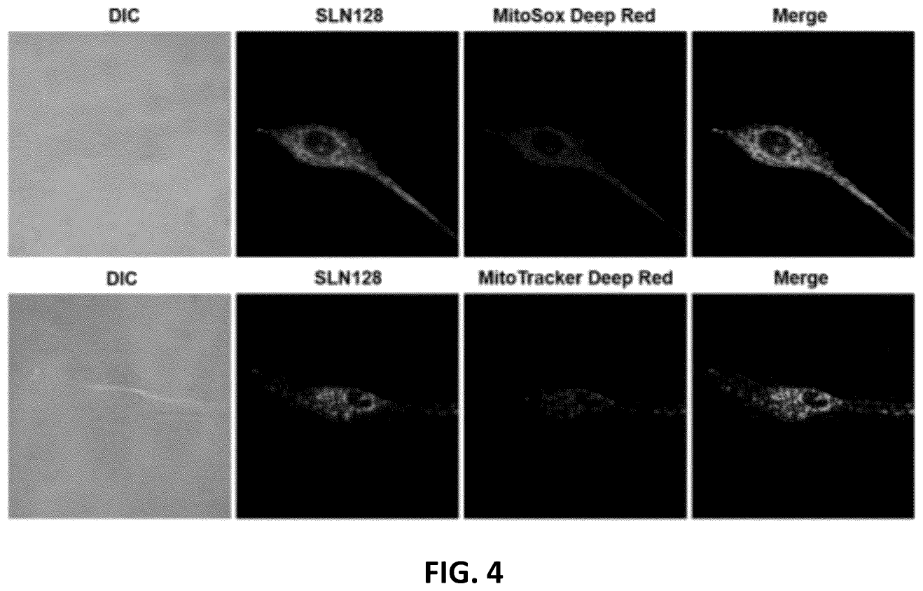

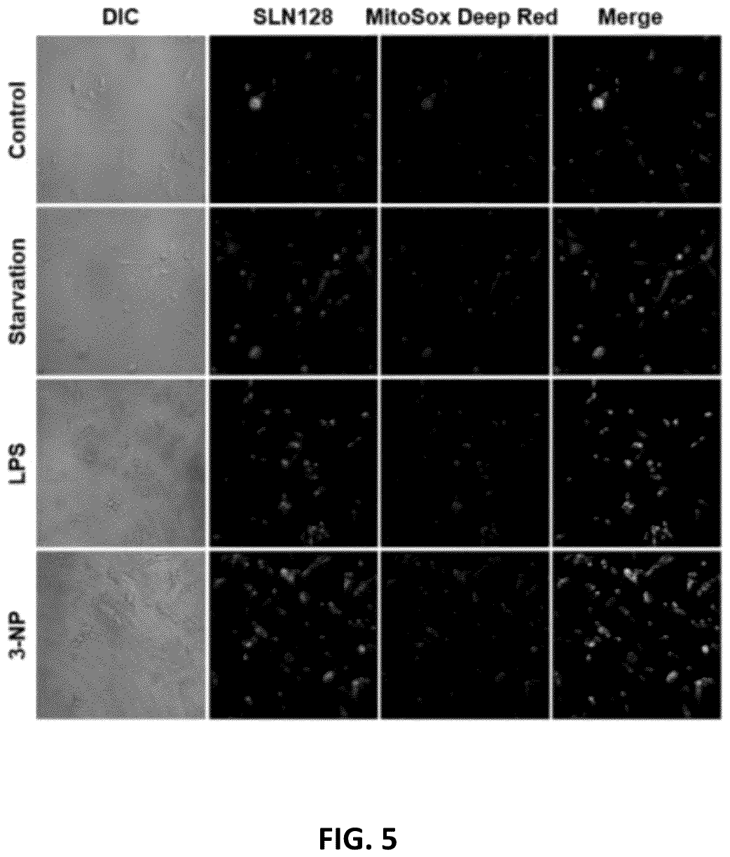

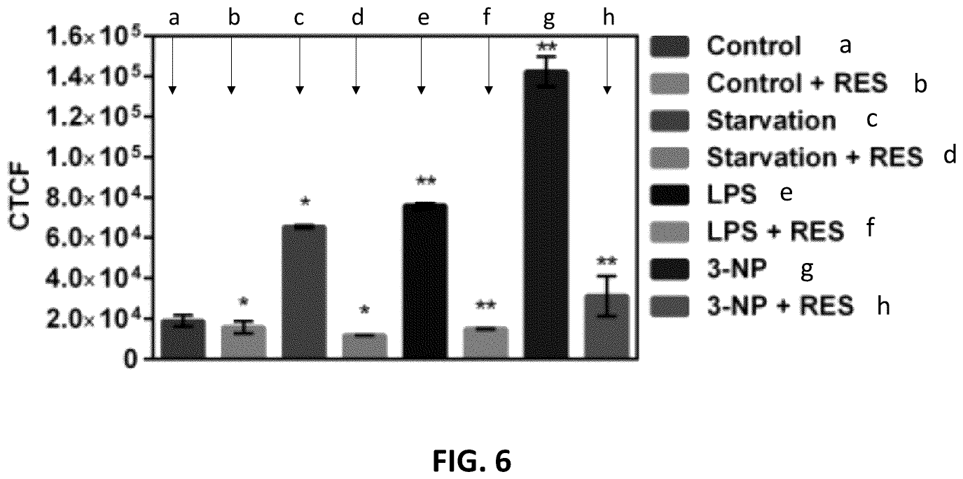

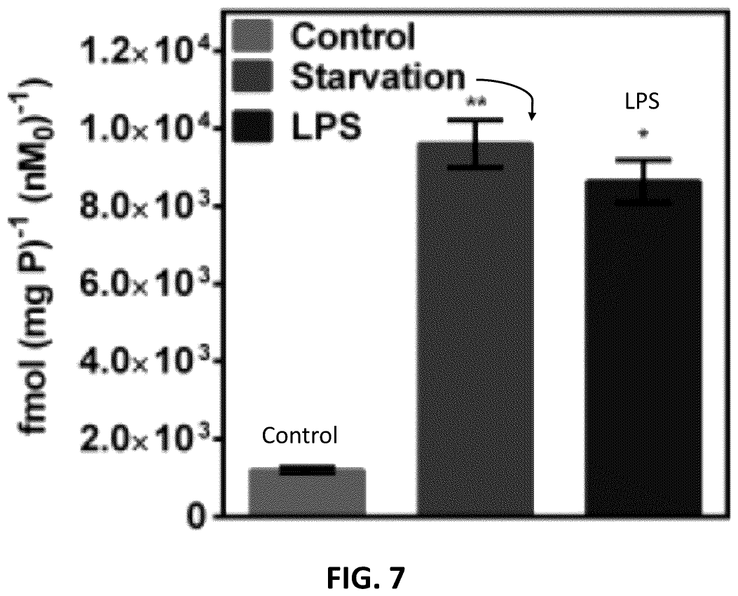

Lipopolysaccharide (LPS), is an endotoxin, released from Gram-negative bacteria. Next, the ability of SLN128 to detect either LPS or 3-Nitropropionic acid (3-NP) induced ROS in human cells was evaluated through live-cell fluorescence imaging. LPS is detected by toll-like receptor 4 (TLR4), a broadly expressed pattern recognition receptor found both on different cell types, including glial cells, and neurons. TLR4 engagement triggers the generation of mitochondrial superoxide from electron transport complex I, which in turn promotes bacterial killing. LPS treatment also triggers increased cytokine and chemokine expression in brain thus driving inflammation. Furthermore, 3-NP is an irreversible inhibitor of mitochondrial succinate dehydrogenase (SDH), which has been used to explore the molecular mechanisms of cell death associated with mitochondrial dysfunction and neurodegeneration. Overall, 3-NP can significantly induce oxidative damage and impair antioxidant defense enzymes in the brain. To this end, intracellular localization of SLN-128 in either the presence or absence of mitotracker deep red (control) was simulataneously assessed in either the LPS or 3-NP stimulated human glioblastoma U87 cells (see e.g., FIG. 4). Single cell fluorescence imaging (100×) indicated that SLN128 indeed penetrates into mitochondria of U87 cells (see e.g., FIG. 4). Further, uptake of SLN 128 in U87 cells was compared under identical conditions with mitoSOX, a probe extensively used for assessing mROS. When compared to untreated U87 cells, LPS- or 3-NP stimulation induced both higher fluorescence of SLN 128, while exhibiting a distinct pattern of probe co-localization with mitoSOX red thus indicating that SLN128 is indeed taken up by superoxide-producing mitochondria (see e.g., FIG. 5). To test, whether SLN128 has ability to monitor therapeutic effects in U87 cells under identical conditions, SLN128 was incubated in either presence or absence of resveratrol (commonly used for mitigating mitochondrial ROS production) under 3 different conditions. The probe uptake was quantified as corrected total cell fluorescence (CTCF) using image J as described earlier. SLN128 demonstrated the ability to monitor resveratrol-induced mitigation of ROS in U87 cells. (see e.g., FIG. 6). For assessing cellular accumulation of ¹⁸F-SLN-128 in U87cells, U87 cells were incubated with ¹⁸F-SLN-128 in either presence or absence of nutrients and LPS (5 μg/mL, for 60 min at 37° C. in the control buffer. Following incubations, cells were assayed for γ-activity as described earlier. The quantified cellular uptake of ¹⁸F-SLN-128 in U87 cells (see e.g., FIG. 7) is reported as fmol ¹⁸F-SLN-128×(nM0)−1×(mg protein)−1. Taken together, ¹⁸F-SLN-128 shows uptake profiles in U87 cells (see e.g., FIG. 7) similar to those observed with fluorescence bioassays (see e.g., FIG. 5 and FIG. 6) indicating a good correlation between fluorescence and radiotracer bioassays. To further assess whether PET counterpart ¹⁸F-SLN-128 administered at tracer concentrations relevant for nuclear imaging demonstrates optimal kinetics (signal/noise ratios) to enable brain imaging in vivo, quantitative biodistribution studies in normal mice (BI6) were performed. Uptake in brain and other critical organs was analyzed in terms of percent injected dose per gram of the tissue (% ID/g).

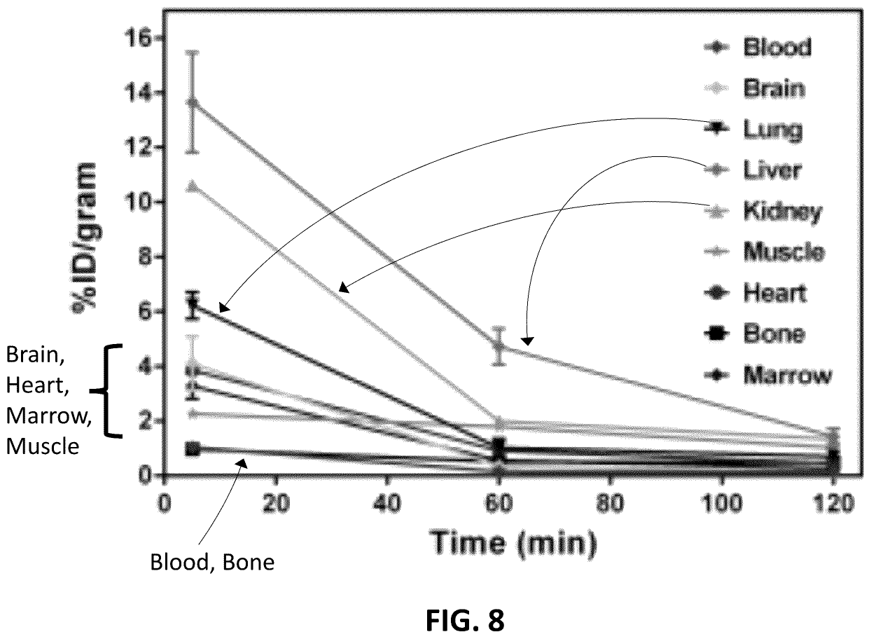

Biodistribution studies with HPLC purified ¹⁸F-SLN-128 in normal mice show transient brain uptake values of 4.11±0.32% ID/g and 0.36±0.01% ID/g, at 5 min and 60 min post tail-vein injection (see e.g., FIG. 8), respectively, thus providing a 5 min/60 min clearance a ratio of 11.4. For comparison, brain uptake ratios of [¹⁸F]-AVID-45 ([¹⁸F]-Florbetapir)(2 min/2 h) and [¹⁸F]-Florbetaben (2 min/4 h) in normal mice are 4.07 (% ID/g (brain): 2 min: 7.33±1.54; 2 h: 1.80±0.07; 7.33/1.80; 4.07) 57 and 5.0 (% ID/g (brain): 2 min: 4.77; 4 h: 0.95; 4.77/0.95; 5.02) 58, respectively. Therefore, the brain uptake clearance ratio of ¹⁸F-SLN-128 (5 min/60 min) is 2.9 and 2.3-fold, superior to [¹⁸F]-AV-45 and [¹⁸F]-Florbetaben, respectively, in healthy mice. Overall, the clearance ratio of 11.4 is 3.3-fold higher than the benchmark (≥3.5) for ability of a given agent to cross the blood-brain barrier.

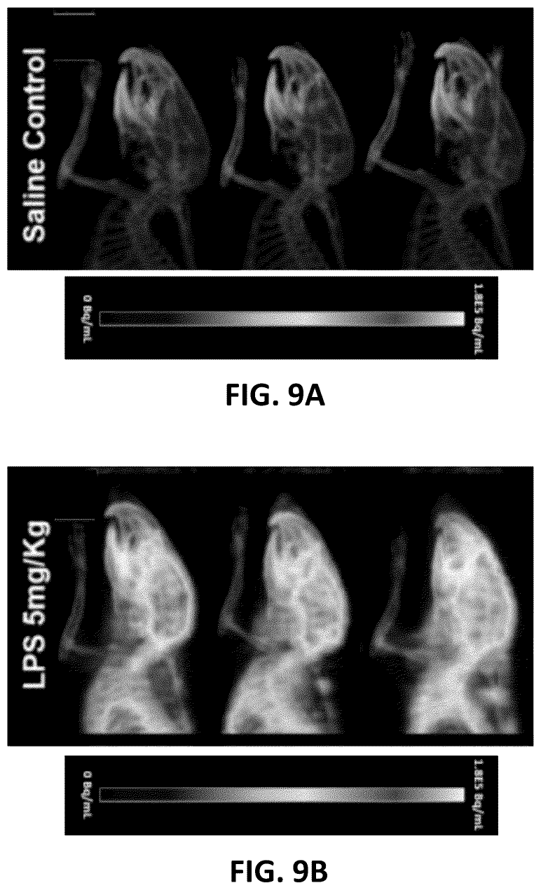

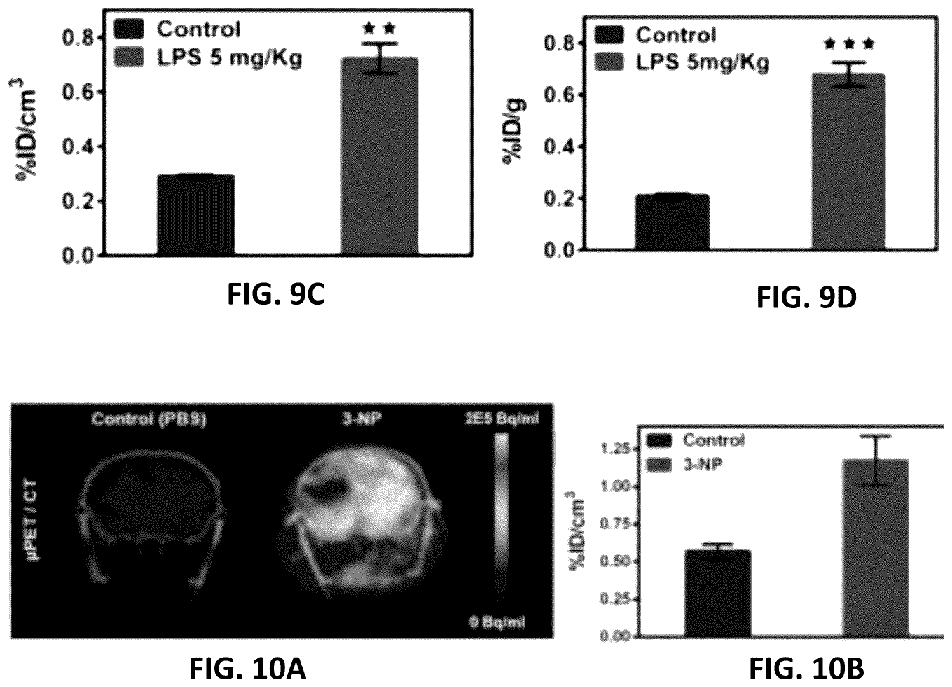

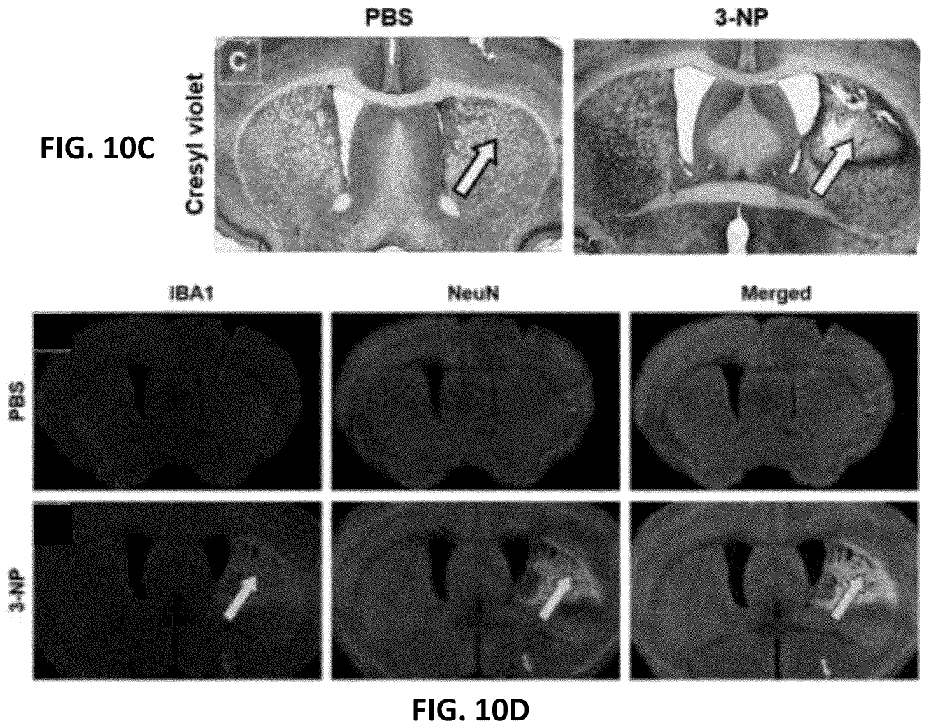

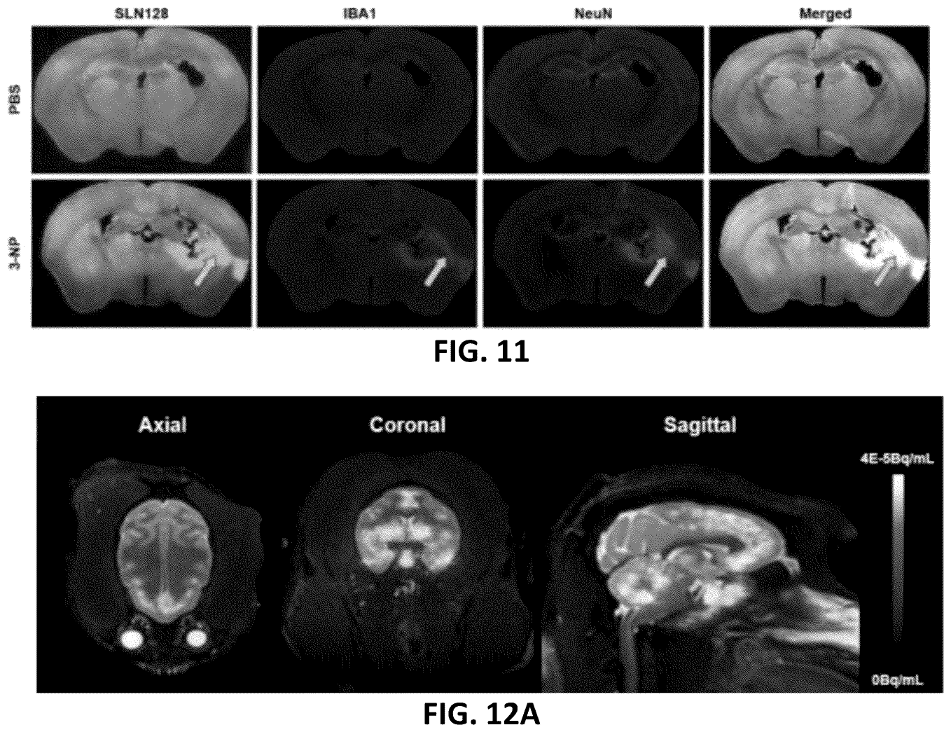

While the clearance ratio of 11.3 (% ID/g; 5 min/60 min) provides evidence for the ability of ¹⁸F-SLN-128 to traverse the BBB in vivo, the agent ¹⁸F-SLN-128 excreted from other critical organs over 1 h (see e.g., FIG. 8); a critical factor likely to result in favorable dosimetry; without defluorination (no bone accumulation) thus consistent with pharmacokinetic profiles of other FDA approved agents. Systemic administration of LPS induces oxidative stress and resulting a release of inflammatory cytokines, sustained microglial activation, and neuronal cell loss in brain areas, such as the hippocampus, cerebral cortex, substantia nigra, ventral tegmental area, and thalamus, alters the levels of neurotransmitters in adult animals, and cerebellum. Thus LPS-induced inflammation provides a useful rodent model for preclinical imaging of ¹⁸F-SLN-128. Preclinical dynamic PET/CT images (summation of frames over 45-60 min) using ¹⁸F-SLN-128 are shown in FIG. 9A-FIG. 9D. Of note, ¹⁸F-SLN-128 demonstrated a 2.5 folds higher uptake in brains of LPS-treated (see e.g., FIG. 9B). Finally, the brain uptake (see e.g., FIG. 9C) derived from PET imaging also correlated with 60 min post imaging quantified uptake (see e.g., FIG. 9D) indicating potential of the radiotracer for imaging endotoxin mediated brain inflammation in vivo. Mitochondrial dysfunction is a major event in the pathogenesis of Huntington disease (HD). It has also been reported that systemic 3-NP administration leads to oxidized proteins in the striatum, as well as a massive loss of striatal neurons. To evaluate potential of ¹⁸F-SLN-128 PET imaging for 3NP-induced ROS-mediated neurodegeneration in mice, PET/CT imaging was performed. Following stereotaxic injection (Bregma coordinates: A/P: 0.2 anterior, M/L: 2 mm right, and D/V: 3.2 mm below cranium) of 3-NP (23 μg; 1 mg/Kg) into stratum of BI6 mice, and 72 h post treatment, tail-vein injection of HPLC purified ¹⁸F-SLN-128 showed 2-fold higher retention in brains of 3-NP treated mice compared with their saline treated counterparts (see e.g., FIG. 10A-FIG. 10D). Finally, unlabeled SLN128 was also injected into 3-NP treated mice post PET imaging (following recovery overnight), brains were extracted, fixed, and sectioned as described (see e.g., FIG. 10A-FIG. 10D), SLN128 maps monoclonal anti-IBA1 and anti-NeuN antibody and localizes in microglial cells and neurons thus indicating molecular specificity (see e.g., FIG. 11).

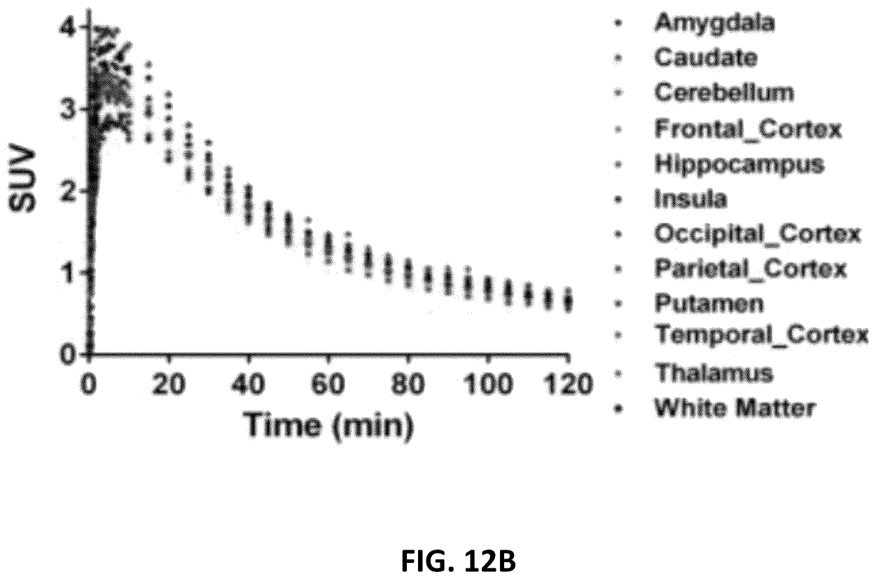

For preliminary evaluation of ability of the tracer to penetrate brain in a nontargeted non-human primate, following intravenous injection of the ¹⁸F-SLN-128, dynamic PET scan of rhesus monkey brains were performed, and co-registered with MR for anatomical reference (see e.g., FIG. 12A-FIG. 12B). ¹⁸F-SLN-128 demonstrated ability to penetrate bloodbrain barrier (BBB) with maximum Standard Uptake Value (SUV) of 3.5 by 7-10 min and excreted from critical regions at later-time points thus pointing to the potential for high signal/noise ratios for investigating ROS-mediated inflammation prevalent in neurodegenerative diseases. Of note, these NHP brain SUV values are 2.5-fold higher than reported for ¹⁸F-ROStrace thus likely to translate into higher contrast brain images compared with ¹⁸F-ROStrace (SUV=1.35). Additionally, samples of venous blood drawn at an interval of 3 min over 2 hours did not show any metabolite, using radio-HPLC and fraction counting using γ-counter. Tissue time-activity curves (TACs) also show fast initial uptake with maximum projection of activity at 7-10 min followed by clearance of ¹⁸F-SLN-128 at later time-points from amygdala, caudate, cerebellum, cortex (frontal, insular, occipital and temporal), cerebellum, hippocampus, putamen, thalamus, and white matter of rhesus monkey brain. Taken collectively, these data indicate that ¹⁸F-SLN-128 uptake could provide a functional imaging of the mitochondrial status within the brain and offers a robust platform for further validation in AD transgenic rodent models, NHPs kinetic studies, and advance into developmental pipeline for performing first-in humans studies.

What is claimed is:

1. A composition comprising a reactive oxygen species (ROS) imaging agent according to any one of Formulas (I-VI)ₐ and (I-VII)_{b}: [symbol]
wherein:
X₁ and X₃ are independently NR¹, O, S, or PR²;
X₂ is N or P;
L is [symbol]
;
n is 0, 1, 2, 3, or 4;
R is H, CH₃, alkyl chain (linear or branched, number of carbons can be 1-4), cycloalkyl (ring size can be 3-7), —OR⁷, NH₂, NHR⁸, NR⁹R¹⁰, SH, SR¹¹, PH₂, PHR¹², PR¹³R¹⁴, halo, CN, NO₂, COOH, COOR¹⁵, CHO, COR¹⁶, CONH₂, CONHR¹⁷, CONR¹⁸R¹⁹, SOR²⁰, SO₂R²¹, SO₃R²², or a chelator core;
Y₁-Y₁₇ are independently CH, CR²³ (wherein R²³ can be alkyl, alkenyl, alkynyl), —OH, —OR²⁴, NH₂, NHR²⁵, NR²⁶R²⁷, SH, SR²⁸, PH₂, PHR²⁹, PR³⁰R³¹, halo, BR³², BR³³R³⁴, CN, NO₂, COOH, COOR³⁴, CHO, COR³⁶, CONH₂, CONHR³⁷, CONR³⁸R³⁹, SOR⁴⁰, SO₂R⁴¹, SO₃R⁴², a chelator core, CO, COO, COS, CONR⁴³, N, NH, NR⁴⁴, NO, O, S, SO, SO₂, BR⁴⁵, or BR⁴⁶R⁴⁷; and
R¹-R⁴⁷ are independently H, halo, C₁-C₁₂ linear alkyl, C₂-C₁₂ linear alkenyl, C₂-C₁₂ linear alkynyl, C₃-C₁₂ branched chain alkyl, C₃-C₁₂ branched chain alkenyl, C₃-C₁₂ branched chain alkynyl and C₃-C₇ cycloalkyl, or (hetero)aryl.

2. The composition of claim 1, wherein R comprises a chelator core selected from NOTA, NODA, DOTA, DTPA, or Triglycine.

3. The composition of claim 1, wherein H is T or ³H.

4. The composition of claim 1, wherein halo is Cl, F, Br, I, ¹⁸F, ⁷⁵Br, ⁷⁶Br, ⁷⁷Br, ¹²³I, ¹²⁴I, ¹²⁵I or ¹³¹I.

5. The composition of claim 1, wherein the ROS imaging agent comprises a chelator core and a metal radionuclide.

6. The composition of claim 5, wherein the metal radionuclide is an ion of gallium-67 (⁶⁷Ga), gallium-68 (⁶⁸Ga), an unlabeled gallium, or a paramagnetic metal which includes an ion of ⁶⁷Ga, an ion of ⁶⁸Ga, an ion of an unlabeled gallium, -indium-111 (¹¹¹In), -iron-52 (⁵²Fe), iron-59 (⁵⁹Fe), -copper-62 (⁶²Cu), -copper-64 (⁶⁴Cu), -thallium-201 (²⁰¹Tl) -technetium-99m (^{99m}Tc), -technetium-94m (^{94m}Tc), -rhenium-188 (¹⁸⁸Re), -rubidium-82 (⁸²Rb), -strontium-92 (⁹²Sr), -yttrium-86 (⁸⁶Y) or yttrium-90 (⁹⁰Y), -zirconium-86 (⁸⁶Zr) or -zirconium-89 (⁸⁹Zr), -aluminium fluoride-18 (Al¹⁸F), a paramagnetic metal ion, a transition metal, or a lanthanide metal ion.

7. The composition of claim 1, wherein the ROS imaging agent comprises a radiolabel or radionuclide.

8. The composition of claim 7, wherein the ROS imaging agent comprises the radiolabel ¹⁸F or ¹¹C.

9. The composition of claim 1, wherein the ROS imaging agent is selected from the group consisting of: 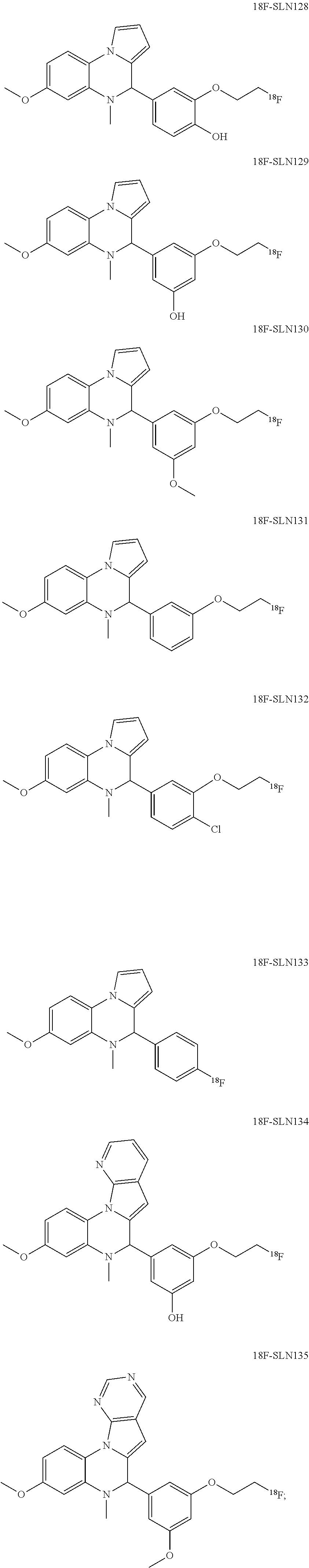
or a pharmaceutically acceptable salt, solvate, polymorph, tautomer, prodrug, or analog thereof.

10. A method of detecting ROS in a subject, the method comprising administering to the subject an effective amount of a composition comprising a reactive oxygen species (ROS) imaging agent according to any one of Formulas (I-VI)ₐ and (I-VII)_{b}: [symbol]
wherein:
X₁ and X₃ are independently NR¹, O, S, or PR²;
X₂ is N or P;
L is [symbol]
;
n is 0, 1, 2, 3, or 4;
R is H, CH₃, alkyl chain (linear or branched, number of carbons can be 1-4), cycloalkyl (ring size can be 3-7), —OR⁷, NH₂, NHR⁸, NR⁹R¹⁰, SH, SR¹¹, PH₂, PHR¹², PR¹³R¹⁴, halo, CN, NO₂, COOH, COOR¹⁵, CHO, COR¹⁶, CONH₂, CONHR¹⁷, CONR¹⁸R¹⁹, SOR²⁰, SO₂R²¹, SO₃R²², or chelator core;
Y₁-Y₁₇ are independently CH, CR²³ (wherein R²³ can be alkyl, alkenyl, alkynyl), —OH, —OR²⁴, NH₂, NHR²⁵, NR²⁶R²⁷, SH, SR²⁸, PH₂, PHR²⁹, PR³⁰R³¹, halo, BR³², BR³³R³⁴, CN, NO₂, COOH, COOR³⁴, CHO, COR³⁶, CONH₂, CONHR³⁷, CONR³⁸R³⁹, SOR⁴⁰, SO₂R⁴¹, SO₃R⁴², chelator core, CO, COO, COS, CONR⁴³, N, NH, NR⁴⁴, NO, O, S, SO, SO₂, BR⁴⁵, or BR⁴⁶R⁴⁷; and
R¹-R⁴⁷ are independently H, halo, C₁-C₁₂ linear alkyl, C₂-C₁₂ linear alkenyl, C₂-C₁₂ linear alkynyl, C₃-C₁₂ branched chain alkyl, C₃-C₁₂ branched chain alkenyl, C₃-C₁₂ branched chain alkynyl and C₃-C₇ cycloalkyl, or (hetero)aryl.

11. The method of claim 10, wherein the ROS imaging agent is preferentially localized to the brain or central nervous system (CNS).

12. The method of claim 10, wherein an imaging signal of the ROS imaging agent is driven by a byproduct mediated by precursors of ROS.

13. The method of claim 10, wherein the subject has or is suspected of having a neurological disease, disorder, or condition.

14. The method of claim 13, wherein the subject has or is suspected of having a preclinical neurological disease, disorder, or condition.

15. The method of claim 14, wherein the preclinical neurological disease, disorder, or condition is selected from amyotrophic lateral sclerosis (ALS), Parkinson's disease (PD), Alzheimer's disease (AD), muscular dystrophy, or multiple sclerosis.

16. The method of claim 10, wherein the subject has or is suspected of having an inflammatory disease.

17. The method of claim 10, wherein the subject has or is suspected of having long-COVID syndrome.

18. The method of claim 10, further comprising exposing the subject to an imaging modality.

19. The method of claim 18, wherein the imaging modality is PET or CT.

20. The method of claim 10, wherein administering the composition comprising the ROS imaging agent results in:
penetration of the ROS imaging agent into a membrane;
oxidation of the ROS imaging agent by ROS; and
trapping of the ROS imaging agent in a cell membrane and intracellular compartments.
